(19) United States
(12) Patent Application Publication
(10) Pub. No.: US 2010/0104621 A1
(43) Pub. Date: Apr. 29, 2010

(54) TREATING ADHD AND OTHER DISEASES INVOLVING INFLAMMATION

(75) Inventor: Ralph E. Waldo, Carmel, IN (US)

Correspondence Address: OVERHAUSER & LINDMAN, 740 W. GREEN MEADOWS DRIVE, SUITE 300, GREENFIELD, IN, 46140, US

(73) Assignee: CONNECTED HEALTH SYSTEMS, LLC, Carmel, IN (US)

(21) Appl. No.: 12/527,110

(22) PCT Filed: Feb. 5, 2008
(86) PCT No.: PCT/US2008/052972

§ 371 Date: Aug. 13, 2009

Related U.S. Application Data
(60) Provisional application No. 60/890,894, filed on Feb. 21, 2007.
(60) Provisional application No. 60/972,039, filed on Sep. 13, 2007.

(51) Int. Cl. A61K 9/70; A61K 31/135; A61K 38/43; A61K 31/675; A61K 31/20; A61K 31/202; A61K 36/00; A61K 36/87; A61P 25/00

(52) U.S. Cl. 424/449; 514/654; 424/94.1; 514/89; 514/558; 514/560; 424/725; 424/766

(57) ABSTRACT

The present disclosure relates to methods and drug delivery systems for treating diseases involving inflammation, including Attention Deficit Hyperactivity Disorder (ADHD) by administering a CNS stimulant to a patient in need thereof so as to maintain steady state serum drug levels that remain therapeutically effective for about 24 and ½ to about 25-27 hours or longer after administration to maintain a constant steady state between doses of medication. The method is employed to restore normal catecholamine levels throughout the day without over-stimulating or under-stimulating the patient. Additionally, the present method of treating inflamτnaτion3 including ADHD, provides, for example, dosing once a day or once a week. The present method also addresses other aspects of the disease, for example, defective P-5-P synthesis, ehminating interleukins and free radicals, and correction of Amino acids, the endocrine system and inflammation. The present systems and methods heal the brain, treat depression and ADHD, and may lower the amount of medication needed over time.

### FIELD OF THE INVENTION

The present invention relates generally to the treatment or prevention of diseases involving inflammation. In particular, the invention pertains to drug delivery systems and methods of treating Attention Deficit Hyperactivity Disorder (ADHD) and other diseases associated with inflammation.

### BACKGROUND

Attention Deficit Hyperactivity Disorder (ADHD) is a genetic disease, often autosomally dominant-inherited mental disorder that affects between 8% to 12% of the population in the United States and around the world. ADHD has roughly equivalent incidence rates in both sexes. ADHD is usually diagnosed in childhood based on symptoms such as hyperactivity, impulsiveness, forgetfulness, mood shifts, and distractibility. ADHD is comprised of three subtypes: (1) predominantly inattentive ADHD; (2) predominantly hyperactive-impulsive ADHD; and (3) combined-type ADHD. While ADHD commonly manifests before age 7, it may go undiagnosed until adolescence or even adulthood. Indeed, some ADHD individuals are able to function quite normally as productive members of society, and even at well-above average levels through effective compensating and/or coping behaviors. This is hardly surprising given the average IQ of ADHD individuals may be 120, well above the average of the general population. About 70% have just the inattentive type and learn to compensate and go unrecognized. Additionally, the hyperactive component disappears as a person ages. Many ADHD patients may not know they have the disease and many become perfectionists. In some cases self-medication is observed in the form of caffeine, nicotine and occasionally alcohol consumption.

Unfortunately, many ADHD patients are not able to compensate adequately, develop low self-esteem, and must receive appropriate treatment in order to achieve their full intellectual and social potential. If left untreated, ADHD may exact a significant hardship on affected individuals, loved-ones, and society at large. ADHD currently has no cure though a number of treatments are available. Additionally, many ADHD patients have been told that their disease would go away with age and that they did not need medication. Genetic disorders do not go away.

Currently, ADHD is believed to be 80% genetic, involving the catecholamine system in the brain and body. Individuals afflicted by ADHD have below normal levels of norepinephrine and dopamine, although these abnormalities may be secondary to defects in other systems such as serotonin receptors, second messenger systems, basic biochemical pathways and co-factors. Evidence suggests that some ADHD individuals display elevated levels of dopamine transporter (DAT) and this may explain their lowered levels of dopamine. Catecholamines occur throughout the body, not just the brain, and imbalances observed in the brain may also occur throughout the body. There is accumulating evidence that catecholamine and indolamine imbalances may be a cause or result of inflammation (G. Ch Beck et al., Crit. Care, 8, 485-491, 2004). Therefore, ADHD may be mechanistically linked with other systems in the body including the immune system, endocrine system, gastrointestinal system and biochemical pathways. As such, an effective treatment for ADHD should address one or more of these underlying biochemical components.

Various studies have addressed the serotonin transporter SLC6A4 and the impact of different alleles on disease states. LL genotype individuals may be more prone to heart disease and SS genotype individuals have a higher incidence of aphthous ulcers. It has also been observed that LL genotypes respond to treatment with Selective Serotonin Reuptake Inhibitors, but SS genotypes respond poorly secondary to tolerability. However, when pindolol is included in the treatment SS individuals respond rapidly. Pindolol acts as an agonist/antagonist at the 5HT1 autoreceptor and increases catecholamine production. The SS genotype has also been shown to increase the risk for addiction, eating disorders, impulsivity, aggression, and misinterpreting cues in the environment as well as being associated with inflammation This receptor is on Chromosome 17 and is located near the gene for Von Recklinghausen's disease. It is well known that Von Recklinghausen's disease is always associated with ADHD. TNF alpha, other cytokines and P38 MAPK have been shown to regulate this receptor. Additionally, defects in the serotonin transporter or receptors have been associated with irritable bowel syndrome. Each receptor that has been shown to cause ADHD, has also been associated with gastrointestinal inflammation although the site of the exact mutation may be different.

Current treatments for ADHD include administering central nervous system (CNS) stimulants such as methylphenidate or amphetamine. A typical treatment regimen for a school-age child involves administering a stimulant in the morning prior to school, and again while at school. Recently-introduced products allow a single dosing of a sustained-release formulation that covers a period of 6-12 hours. Long-acting stimulant products avoid the necessity of dosing two or more times per day. Current treatments generally leave a child un-dosed or under-dosed in the late afternoon and evening hours. Limiting treatment to 6 to 12 hours per day has been viewed as necessary to avoid undesirable side effects such as insomnia or loss of appetite. Unfortunately, current treatment regimens do not correct the catecholamine imbalance over the entire day and allow the patient to revert to an unbalanced state for as much as 12 to 16 hours per day (i.e. 24 hour period). Thus, there remains a need for a better treatment for ADHD to allow a patient to have a consistent level of catecholamines around the clock, thereby returning their brain and body to normal.

### SUMMARY OF THE INVENTION

It is an object of the present invention to provide a method for treating and/or preventing diseases that may be associated with inflammation and/or catecholamine imbalance by administering one or more CNS stimulant(s) such that a steady state, therapeutically-effective serum level of said stimulant(s) is maintained substantially around the clock. In one embodiment, the extended treatment period allows for the medication to wear off while the medication taken the next day is being absorbed so that a steady state can be maintained consistently. Just as insulin for only 4 to 12 hours would be ineffective and would not fully treat diabetes, providing one or more CNS stimulant(s) for only 4 to 12 hours does not maintain a consistent level of catecholamines

It is another objective of the present disclosure to provide a method for treating ADHD by administering one or more CNS stimulant(s) such that steady state therapeutically effective serum levels of stimulant are maintained substantially around the clock following administration.

It is a further object to provide a method for treating ADHD by co-administering one or more CNS stimulant(s) with an anti-inflammatory or other agent(s) such that therapeutically effective serum levels of stimulant are maintained substantially around the clock following initial ingestion.

It is a further object to normalize catecholamine levels in ADHD patients by administering one or more CNS stimulant(s) to maintain therapeutically effective serum levels substantially around the clock.

It is a further object to normalize catecholamine levels in ADHD patients by co-administering one or more CNS stimulant(s) with an anti-inflammatory or other agent(s) to maintain therapeutically effective serum levels of said stimulant(s) substantially around the clock.

It is a further object to provide a method of treating ADHD patients by administering an appropriate dose of one or more CNS stimulant(s) such that steady state therapeutically effective serum levels of stimulant are maintained substantially around the clock, while maintaining normal vital signs including heart rate and blood pressure. Catecholamines, which are present throughout the body, modulate heart rate and blood pressure. If the dose of a stimulant is too high, vital signs typically go up. Current treatment regimens for ADHD may allow vitals signs to go up and deliver different amounts of medication during the day. One of the problems with current treatment regimens is that they may result in too much medication being given and do not maintain a consistent steady state of medication. This could harm the heart and cardiovascular system over time. Moreover, too much norepinephrine has been shown to increase IL-1, IL-6 and TNF alpha which are inflammatory.

It is a further object to effectively treat ADHD patients substantially around the clock by administering an appropriate dose of one or more CNS stimulant(s) without disrupting normal sleep patterns. If catecholamines are returned to a normal physiological level a person will sleep better as their body is normalized, have better airflow, and a normal sleep EEG. Norepinephrine is required to release adequate amounts of melatonin. In addition, growth hormone levels should increase especially before awakening and cortisol levels should decrease. Finally, restless legs or periodic limb movements and sleep hygiene are expected to improve. All of these changes should improve overall health and by giving the medications around the clock may allow the brain to be repaired. Catecholamines release BDNF, which will grow new brain cells. However, if this is not done around the clock, healing will be difficult. The current stimulant medications do not cover the period of time when a person is sleeping.

It is a further object of the present invention to treat or prevent diseases associated with inflammation by administering pyridoxal 5′-phosphate (P-5-P or PLP) the active co-factor form of vitamin B6 alone or in combination with one or more anti-inflammatory agent(s), or in combination with one or more CNS stimulant(s). Many people suffering from ADHD have defects in vitamin B6 synthesis, which may be genetic, environmental, feedback mechanisms and an imbalance or deficiency in co-factors for proper synthesis of vitamin B6. Vitamin B6 is required as a co-factor for over 100 enzymes in the body and is involved in catecholamine, indolamine and GABA synthesis. It also is a co-factor for melatonin synthesis, amino acid pathways, blood cell differentiation and formation, fatty acid synthesis and endocrine hormone synthesis. It also allows people to make their own neurotransmitters, blood cells, hormones, fatty acids, amino acids and is an antioxidant and anti-inflammatory. Additionally, giving back dopamine can decrease the activity of pyridoxal kinase, the enzyme which makes P-5-P or PLP. Thus, it is important to give back only what is needed in terms of dopamine to ensure that a patient does not turn off their own production of P-5-P through feedback inhibition. If P-5-P is decreased, this could impact 100 pathways in the body. Homocysteine has been shown to increase in Parkinson's Disease patients treated with L-dopa and PLP prevents this. If catecholamines are normal this should not happen, but this is protective and also should allow a person with a defect in PLP syntheses to make their own catecholamines and normalize other pathways impacted by ADHD or a lack of PLP.

It is another object of the present invention to treat ADHD by administering pyridoxal 5′ phosphate (P-5-P or PLP) alone or in combination with a CNS stimulant(s) such that said stimulant(s) is maintained at therapeutically effective serum levels substantially around the clock. Dopamine and norepinephrine are known to suppress the activity of pyridoxal kinase by a feedback mechanism. This would turn off the production of P-5-P thereby suppressing up to perhaps 100 other pathways in the body. By giving back only the appropriate level of catecholamines this should be minimized, but if it does occur, it is important to keep the other pathways intact to prevent disease.

It is another object to treat an ADHD patient by administering one or more CNS stimulant(s) via once per week transdermal patch delivery, such that therapeutically effective serum levels of stimulant are reached within about 30 minutes to about 3-6 hours after attaching the patch, and thereafter maintained at steady state throughout the next 6 days, and wherein on day 7 said therapeutically effective serum levels of stimulant decline below effective levels. A new patch can be applied on Day 7 to maintain a consistent steady state serum level of stimulant.

It is another object to prevent or treat a disease(s) or condition(s) that may co-occur with or otherwise be associated with ADHD including, for example, diabetes, metabolic syndrome, autoimmune disease, dementia, gastrointestinal inflammation, headaches and cancer by administering a CNS stimulant, or a stimulant plus an anti-inflammatory agent so as to maintain therapeutically effective levels of stimulant, or stimulant and anti-inflammatory agent, substantially around the clock.

It is another object to provide a convenient dosage form for administering to ADHD patients comprising one or more CNS stimulants, optionally also including an anti-inflammatory agent, wherein said dosage faun releases the active agent(s) to maintain therapeutically effective levels of stimulant, or stimulant and anti-inflammatory agent substantially around the clock.

It is another object to provide a pharmaceutical dosage form comprising one or more CNS stimulants, optionally also including an anti-inflammatory agent and/or pyridoxal 5′ phosphate at therapeutically effective levels such that effective levels of stimulant are maintained substantially around the clock. In one embodiment, when one dosage of medication is wearing off, a second dose of the medication would be absorbed to maintain steady state.

In accordance with these and other objectives, one embodiment of the invention relates to a method for treating and/or preventing a disease that may be associated with inflammation comprising administering about 5 mg to about 400 mg of a CNS stimulant(s), optionally also including one or more anti-inflammatory agent(s), one or more times per 24 hour period such that a therapeutically effective serum level of stimulant(s) is reached within about 30 minutes to about 4 hours following administration and thereafter maintained at steady state substantially around the clock.

Another embodiment relates to a method for treating and/or preventing a disease that may be associated with inflammation comprising administering about 5 mg to about 400 mg of a CNS stimulant(s), optionally also including co-administering pyridoxal 5′ phosphate (P-5-P or PLP), one or more times per 24 hour period such that a therapeutically effective serum level of stimulant(s) is reached within about 30 minutes to about 4 hours following administration and maintained thereafter at steady state substantially around the clock.

Another embodiment relates to a method for treating and/or preventing a disease associated with inflammation comprising administering pyridoxal 5′ phosphate (P-5-P or PLP), optionally also including co-administering one or more anti-inflammatory agent(s).

Another embodiment relates to a method for treating ADHD comprising administering between 5 mg to 400 mg of a CNS stimulant(s) one or more times per 24 hour period, such that a therapeutically effective serum level of stimulant is reached within about 30 minutes to about 4 hours following administration and maintained at steady state substantially around the clock.

Another embodiment relates to co-administering one or more CNS stimulant(s) and one or more anti-inflammatory agent(s) to treat ADHD such that therapeutically effective serum levels of stimulant(s) are reached within about 30 minutes to about 4 hours following administration and maintained at steady state substantially around the clock.

Another embodiment of the present invention relates to co-administering about 5 mg to about 400 mg of one or more CNS stimulant(s) and pyridoxal 5′ phosphate (P-5-P or PLP), to treat ADHD such that a therapeutically effective serum level of stimulant(s) is reached within about 30 minutes to about 2-4 hours following administration and maintained thereafter at steady state substantially around the clock.

Another embodiment relates to once-weekly transdermal patch delivery of a CNS stimulant(s), optionally also including administration of one or more anti-inflammatory agent(s), to an ADHD patient wherein an initial pulse of stimulant is released within about 30 min to about 4 hours after application, followed thereafter by one or more additional delayed square wave, or pulsed-dose, releases of drug such that a therapeutically effective, steady state serum level of stimulant is maintained substantially around the clock over a period of one week following initial application.

Another embodiment relates to treatment or prevention of a disease or condition associated with ADHD comprising administering one or more CNS stimulants and optionally one or more anti-inflammatory agent(s), so as to maintain therapeutically effective levels of stimulant substantially around the clock.

Another embodiment relates to treatment or prevention of a disease or condition associated with ADHD comprising administering one or more CNS stimulants and optionally pyridoxal 5′ phosphate, so as to maintain therapeutically effective levels of stimulant substantially around the clock.

Another embodiment relates to treatment or prevention of a disease or condition associated with ADHD comprising administering pyridoxal 5′ phosphate.

Another embodiment relates to suitable dosage forms including, for example, a tablet, capsule, or skin patch for delivering a CNS stimulant(s), or CNS stimulant(s) plus anti-inflammatory agent(s), or a CNS stimulant(s) plus pyridoxal 5′ phosphate, or combination thereof, to a patient in need thereof, wherein an initial pulse of stimulant is released within about 30 min to about 2-4 hours after ingestion or application, followed thereafter by one or more additional delayed releases of stimulant(s), optionally also delayed release of anti-inflammatory agent and/or P5P, such that a steady state, therapeutically effective serum level of stimulant(s), optionally also anti-inflammatory and/or P-5-P or PLP, is maintained substantially around the clock following ingestion. This extended treatment period improves a patient's ability to sleep and results in better regulation of the endocrine system and inflammation.

Another embodiment relates to the use of CNS stimulant(s) for the manufacture of a medicament which provides steady state, therapeutically effective serum levels of stimulant substantially around the clock for the treatment of ADHD.

These and additional features of the disclosure will become apparent to those skilled in the art upon consideration of the following detailed description of the illustrative embodiments.

### BRIEF DESCRIPTION OF THE DRAWINGS

FIG. 1: shows an idealized target square-wave plasma profile of a stimulant comprising a rapid initial rise within about 30 minutes to about 2-4 hours after administration, followed thereafter by a steady state plateau that remains within the therapeutically effective range for about 24 and ½ to 25-27 hours or longer following administration, and thereafter drops below the therapeutically effective range.

FIG. 2: shows an idealized target pulsed-wave plasma profile of a stimulant comprising a rapid initial rise within about 30 minutes to about 2-4 hours after administration, followed thereafter by successive delayed releases or pulses P1, P2, and P3 that maintain a wave-like steady state profile that remains within the therapeutically effective range for about 24 and ½ to 25-27 hours or longer following administration, and thereafter drops below the therapeutically effective range.

FIG. 3: shows an idealized target plasma profile for a once per day dosage form that delivers steady state therapeutically effective levels of a CNS stimulant and optionally also including P-5-P or PLP, substantially around the clock; a first immediate release (IR) discharges stimulant and optionally P-5-P or PLP essentially immediately, followed by second (PR1) and third (PR2) longer-acting delayed releases of stimulant that extend out to about 24 to about 26 hours. The immediate release component of the next day's dosage is represented by IR2.

FIG. 4: shows a graphical representation of a target dissolution profile of a CNS stimulant formulation according to the invention.

### DETAILED DESCRIPTION

For the purposes of promoting and understanding the principles of the invention, reference will now be made to one or more illustrative embodiments and specific language will be used to describe the same. It will nevertheless be understood that no limitation of the scope of the invention is thereby intended.

The present disclosure relates to pharmaceutical drug dosage forms, drug delivery systems, and methods for treating diseases involving inflammation, for example ADHD, by administering one or more suitable CNS stimulant(s) substantially around the clock. Such treatment is expected to restore normal levels of catecholamines at steady state over sustained time periods, i.e. substantially throughout the 24 hour period of each treatment day. In one embodiment the present invention relates to treating patients with ADHD, and/or individuals from families in which there is at least one ADHD affected individual. ADHD patients and members of their family may be predisposed to developing other diseases including obesity, breast cancer, prostate cancer, melanoma, pancreatic cancer, colon cancer, stomach cancer, liver cancer, Lung cancer, leukemia, lymphoma, osteosarcoma, pituitary tumors, meningiomas, glioblastomas, medulloblastomas, renal cell carcinoma, endometrial cancer, ovarian cancer, polycystic ovarian disease, testicular cancer, thyroid cancer, retinoblastoma, bladder cancer, uterine cancer, macular degeneration, seizures, cardiac arrhythmias, cardiovascular disease, peripheral vascular disease, aneurysms, strokes, heart failure, hypertension, hypercholesterolemia, diabetes, rheumatoid arthritis, osteoporosis, systemic lupus erythmatosis, autoimmune diseases, thyroid disease, Von Willenbrand's disease, blood disorders, Multiple Myeloma, certain forms of deafness, cataracts, gallbladder disease, appendicitis, Asthma, allergies, COPD, Parkinson's disease, Multiple sclerosis, frontotemporal dementia, Lewy body dementia, Alzheimer's dementia, Fibromyalgia, Chronic Fatigue syndrome, Migraine headaches, PMS, PMDD, Huntington's disease, Creutzfeld Jacob disease, Familial tremor, apthous ulcers, esophagitis, gastritis, GERD, stomach ulcers and motility issues, irritable bowel syndrome, Celiac disease, malabsorption, leaky gut syndrome, Ulcerative Colitis, Crohn's disease, pancreatitis, esophagitis, tonsillitis, recurrent infections, otitis media, eczema, psoriasis, dermatitis, osteoarthritis and joint injury, connective tissue diseases, autism, dyslexia, learning disabilities, failure to thrive and grow, testicular failure, endometriosis, abnormal uterine bleeding, premature ovarian failure, tooth and gum disease, connective tissue diseases, joint disease and weakness, muscular dystrophy, susceptibility to chronic infection, acidemia, Guillane Bare, failure to clear viruses, bacteria and fungus, yeast infections, Candida in the GI system, Restless Legg Syndrome, Narcolepsy, Periodic Limb movement disorder, Circadian Rhythm Disorder, Seasonal Affective Disorder, Major Depressive Disorder, Dysthymia, Anxiety disorders, substance abuse disorders, impulse control disorders, oppositional behavior, Enuresis, sleep walking, night terrors, sudden death and other endocrine, inflammatory and gastrointestinal disorders. The present invention is expected to be useful for treating and/or reducing the risk of developing these and other diseases that may be associated with ADHD. It is believed that treating patients with an effective dose of stimulant substantially around the clock at steady state will reduce these risks. It is believed that the catecholamines and Vitamin B6 synthesis are commonly involved in the pathophysiology of inflammation and the development of numerous diseases.

ADHD affects children and adults and is associated with lower than normal levels of dopamine, norepinephrine and other neurotransmitters. Unlike previous methods for treating ADHD in which stimulants were administered for periods ranging from about 4 hours to about 12 hours, the present invention relates to methods in which therapeutically effective serum levels of stimulant are maintained substantially around the clock. Prior treatment methods were based on the belief that providing a stimulant for greater than about 12 hours per day would lead to undesirable behavior and side effects including, for example, over-stimulation, loss of appetite, and inability to sleep at night. Surprisingly, the present invention has found that restoring normal catecholamine levels in an ADHD patient substantially throughout a 24 hour period is more effective in treating ADHD and in reducing undesirable side effects. It is believed that the present methods also reduce the risk of developing other diseases associated with catecholamine imbalance and inflammation.

The methods of the invention comprise administering CNS stimulant(s) to treat ADHD such that therapeutically-effective serum levels of stimulant are maintained at a steady state substantially around the clock. It is desirable to maintain a steady state serum level of stimulant that normalizes the patient substantially around the clock. Preferably, the duration of action terminates as the next medication dose is peaking so that a steady state is continuously maintained. Administering a stimulant about every 24-36 hours would allow a routine to develop and make taking the medication more convenient for patients suffering from ADHD.

As used herein “ADHD” or “ADD” refers to a child or adult having Attention Deficit Hyperactivity Disorder, or Attention Deficit Disorder including the subtypes: predominantly inattentive ADHD, predominantly hyperactive-impulsive ADHD, and combined-type ADHD. A diagnosis of ADHD or ADD would ordinarily be made by a qualified physician with a detailed interview and possibly using one or more acceptable diagnostic tests and rating scales, or criteria from DSM-IVr. As used herein ADHD and ADD is also applied to individuals who may not meet one or more acceptable diagnostic tests but who display biochemical and/or physiological symptoms of the disease including, for example, below normal levels of neurotransmitters and/or catecholamines, and/or below normal levels of amino acid precursors to neurotransmitters including, for example, tryptophan, phenylalanine and tyrosine. This latter group of ADHD patient is often adept at compensating behaviors that may go undetected by currently available diagnostic tests. Such ADHD patients are intelligent and often do not meet hyperactive or impulsive criteria and are not diagnosed. Physicians have not been fully trained to recognize or diagnose this disease and are reluctant to treat it secondary to the many associated myths and misconceptions.

As used herein the term “co-administer” refers to administering one or more CNS stimulant(s) and one or more other agents, for example, anti-inflammatory agent(s) and/or pyridoxal 5′ phosphate (P-5-P or PLP), sequentially, concurrently, or simultaneously. Co-administration may involve administering agents separately or as a single composition to a subject in need thereof. If administered sequentially, the period between administration of stimulant and other agent(s) may, for example, be between 6 to 12 hours. Pharmaceutical formulations and dosage forms can comprise a stimulant(s) and anti-inflammatory agent(s), or a stimulant(s) and pyridoxal 5′ phosphate, or any combination thereof, and optionally also include one or more pharmaceutically acceptable excipients.

The term “immediate-release” means that greater than about 50%, alternatively greater than about 75%, or alternatively, substantially all of an active pharmaceutical agent is released within about 30 minutes to about 4 hours; preferably between about 30 minutes to about 3 hours; most preferably between about 30 minutes to about 2 hours following ingestion or administration of the agent.

The term “normal” or “normalize,” as applied to restoring catecholamine levels in an ADHD patient, refers to elevating dopamine and/or norepinephrine levels to physiologically normal levels (i.e. within a range typical of non-ADHD individuals), such that the patient is able to function normally without being under-stimulated or over-stimulated as assessed by the patient, a parent, a school teacher, a physician, or other appropriate person, or by application of any other suitable test including scanning techniques such as neurometrics, PET scans, FMRI, or SPECT scans to detect catecholamine levels or serum levels of catecholamines. Serum or urine catecholamine levels may also assist in determining appropriate levels, depending on the age of the individual. Vital signs should not be elevated when treated appropriately and patients should sleep better and perform better on tasks. ADHD rating scales can also assist in determining normal functioning and determining the best dose for a given patient. A TOVA or other psychological test can also assist in determining a person's optimal dose.

As used herein, the phrase “target square-wave profile” or “square wave profile” refers to plasma levels of a stimulant, in which a relatively rapid initial rise occurs within about 30 minutes to about 2-4 hours after administration, followed thereafter by a steady state plateau that remains within the therapeutically effective range up to substantially around the clock, for example, for about 24 and ½ to about 36 hours following administration, and thereafter drops below the therapeutically effective range. Preferably, the therapeutically active range is maintained for a period of about 18 hours to about 36 hours, more preferably for about 24 hours to about 36 hours, most preferably for about 24 and ½ to about 25-27 hours or more following administration.

The term “steady state” as used herein refers to serum levels of an active pharmaceutical or nutritional agent, e.g. a CNS stimulant or vitamin, wherein equilibrium plasma levels of the active agent is achieved when the amount of the agent being eliminated from the body is equal to the amount administered. In general, steady state is achieved after four and one-half to five half-lives of the given agent have elapsed. Dosing interval and agent half-life are relevant to the accumulation of an agent in the body and achievement of steady state.

As used herein the term “stimulant” or “CNS stimulant” refers to a central nervous system stimulant. A variety of stimulant compounds are suitable for use according to the present invention including but not limited to methylphenidate and all chemical and chiral derivatives and salts thereof, and amphetamine, amphetamine base, and all chemical and chiral derivatives and salts thereof. In addition, a number of commercially available stimulant products are suitable for use according to one or more embodiments of the present invention including, for example, Ritalin®, Focalin®, Adderall®, and Dexedrine to name a few.

As used herein, the term “substantially around-the-clock” or “substantially around-the-clock dosing” refers to a substantially continuous period of dosing. For example, dosing for about 24 hours a day; dosing for about 20 hours to about 36 hours or more. For example, substantially around-the-clock entails dosing for about 48 hours, about 72 hours, about 96 hours, about 120 hours, about 144 hours, or about 168 hours, or about 2 weeks, about 3 weeks, or about 4 weeks. Preferably the phrase refers to about 22 hours to about 30 hours; more preferably about 23 hours to about 26 hours; more preferably still about 24 hours to about 25 hours, still more preferably about 24 to about 26 hours, or about 24 to about 27 hours; most preferably about 24.5 hours to about 25-27 hours

The term “sustained-release” refers to long-acting dosage forms for administering an agent such as a pharmaceutical drug or nutritional agent, e.g. a CNS stimulant. Sustained release systems may refer to square-wave release in which an initial quick release is followed by a continuous slower release in which serum levels of the active agent are maintained more or less at a steady state within the therapeutically active window. Sustained release may also be achieved through pulsed, or periodic release dosage forms. The term “sustained-release” is used in its conventional sense to refer to a drug formulation that provides for gradual release of a drug over an extended period of time, that preferably results in substantially constant blood levels of the agent over an extended time period such as up to about 20 to 24 hours or more. Sustained-release also entails longer periods, e.g. about 48 hours, about 72 hours, about 96 hours, about 120 hours, about 144 hours, and about 168 hours after drug administration.

The term “delayed-release” is used in its conventional sense to refer to a drug formulation that provides for release of a drug after administration that includes, for example, a delay of release of up to about 1 hour, about 2 hours, about 3 hours, about 4 hours, about 5 hours, about 6 hours, about 7 hours, or about 8 hours or more after administration.

The term “pulsatile-release” is used in its conventional sense to refer to a drug formulation that provides release of drug in such a way as to produce pulsed plasma profiles following administration of the drug.

By the term “transdermal” drug delivery is meant delivery by passage of a drug through the skin or mucosal tissue into the bloodstream.

“Therapeutically-effective” as used herein refers to treatment of patients to achieve a desired clinical or therapeutic benefit, i.e. to minimize, reduce, or eliminate a patient's untreated symptoms. For example, ADHD patients experience reduction of hyperactivity, boredom, impulsiveness, forgetfulness, procrastination, misplacing items, lack of efficiency, poor sleep hygiene, mood shifts, interest-based attention, and distractibility to name a few. Therapeutically-effective may also entail preventing or reducing the risk of developing diseases or conditions associated with chronic or acute inflammation. This objective is achieved by administering one or more CNS stimulant(s), optionally also including administering or co-administering one or more anti-inflammatory agent(s), or PLP or P5P to maintain an effective steady-state serum concentration of stimulant(s) substantially around the clock. In certain illustrative embodiments, the term may be applied to periods of time greater than one about 24 hour period, for example, for 2, 3, 4, 5, 6, or 7 days, or for longer periods including, for example, 2, 3, or 4 weeks.

The terms “P-5-P or PLP”, “P-5-P”, and “PLP” refer to pyridoxal 5-phospate, the active co-factor form of vitamin B6. In one embodiment, the methods according to the invention contemplate administering P-5-P or PLP alone or in combination with CNS stimulant(s) one or more times per day, or one or more times per week. There are many forms of vitamin B6 and multiple steps are involved in synthesizing P-5-P or PLP. Many patients with ADHD have defects in synthesizing P-5-P or PLP, which is needed to make neurotransmitters and regulate hormone and other biochemical pathways involved in amino acid, lipid, and endocrine pathways. Genes involved in the synthesis of P-5-P are located in close proximity to genes that are associated with ADHD and this may explain observed aberrancies in the status of P-5-P in ADHD patients, along with alterations in chemicals and co-factors that can impact the synthesis of P-5-P. However, giving back dopamine can inhibit pyridoxal kinase and shut down up to as many as 100 pathways in the body secondary to not having enough P-5-P. Co-administering P-5-P and a stimulant may allow a patient to avoid feedback inhibition and enable them to make their own catecholamines, while also enabling other important biochemical pathways to continue to function. If a patient is deficient in P-5-P prior to treatment, P-5-P administration should help these pathways function better. Returning a patient to normal levels of catecholamines is expected to prevent feedback inhibition of pyridoxal kinase as a person is physiologically normal.

The term “therapeutically-effective dose” or “therapeutically-effective dosage” refers to the dosage of stimulant(s) and/or other agent(s) administered to an ADHD or other patient or individual that achieves an optimal clinical benefit for the patient including, for example, in an ADHD patient reduction in hyperactivity, impulsiveness, forgetfulness, distractibility, improvement in ability to concentrate, improved ability to perform on the job or at school, improved social skills and behavior, reduced inflammation, reduced risk for developing one or more chronic diseases associated with inflammation. A therapeutically-effective dose is determined by the medical practitioner according to the response of the patient to treatment. For example, a practitioner would generally titrate a therapeutically-effective dose by administering increasing dosages of stimulant until an optimal response is achieved, the objective being to find a dose that does not under-stimulate or over-stimulate the patient. There could be substantial variance among patients. However, in general, a therapeutically-effective dose for an amphetamine-based stimulant is expected to be in the range of about 5 to about 100 mg per 24 hour period. For methylphenidate stimulants, a therapeutically-effective dose is generally expected to be in a range of about 5 to about 400 mg per 24 hour period. In all cases a therapeutically-effective dose is intended to be maintained substantially around the clock at steady state.

Generally, people respond preferentially to either a methylphenidate product or derivative, or an amphetamine product or derivative. The product that is appropriate for an individual will release beta phenylethylamine (PEA). The medication that is inappropriate will not increase PEA. Amphetamines may enter target cells and release catecholamines along with blocking their re-uptake. Methylphenidate blocks the reuptake of catecholamines. Both medications exhibit some monoamine oxidase (MAO) inhibition. Each medication has different effects on urinary catecholamines and breakdown products. If the wrong medication or dose is administered to a patient they may feel tired, irritable, jittery, have elevations in their vital signs, retain fluid, or feel hyperactive, and not get the expected benefits. In contrast, administering the appropriate medication at the proper dose is expected to improve the patient's overall wellbeing and ability to function.

### Attention Deficit Hyperactivity Disorder (ADHD) and the Inflammatory Response

ADHD affects people of all ages and is frequently detected during childhood. It presents in different ways and is categorized as: (1) predominantly inattentive ADHD; (2) predominantly hyperactive-impulsive ADHD; and (3) combined-type ADHD. The underlying cause of ADHD remains unknown, although it appears to involve the frontal lobes, the basal ganglia, and the central aspects of the cerebellum. SPECT scans have revealed that ADHD patients have different blood flow patterns, and PET scans have revealed higher levels of dopamine transporters in the striatum and a reduced amount of glucose utilization by the brain when a patient is focusing.

ADHD definitely involves a genetic component including a gene that encodes a dopamine transporter, specifically the 10-repeat allele of the DAT1 (SLC6A3) gene on chromosome 5p, and the 7-repeat allele of the DRD4 gene on chromosome 11p15. There is also some evidence for an association between ADHD and the dopamine beta hydroxylase gene, DBH on chromosome 9q. 5HT2A, on chromosome 13 at 13q14-q21, is also presently being tested on a family in treatment and is now being confirmed as a cause of ADHD. Additionally, SLC6A4 on chromosome 17 at q11.2-q12 and more specifically the SS genotype has been reported along with, SNAP25, 5HTR1B, DRD1 (5q), and COMT on 22q11. Other genetic associations are ADHD1 on chromosome 16p13, ADHD2 on chromosome 17p11, and ADHD3 on chromosome 6q12. Numerous other genes encoding second messengers, transcription factors, co-factors along with inflammatory pathways are all likely involved in the genotype for ADHD. One example of a candidate gene is TFAP2B, a transcription factor on chromosome 6 p12-21.1. Defects in this gene have also been associated with Sudden Infant Death Syndrome, intestinal inflammation and breast cancer.

The inflammatory response is of interest in understanding ADHD. ADHD may be a response to, or cause of, inflammatory disease associated with abnormal neurotransmitter levels found throughout the body. ADHD has been observed a patient with Severe Combined Immunodeficiency Syndrome (SCID) and short stature. SCID can be caused by a defect in the IL-7 receptor, which has been mapped to Chromosome 5p13. Interestingly, the ADHD4 gene also maps to region 5p13 as do genes that are associated with short stature, hypertension and growth hormone receptor. IL-7 is now being associated with Multiple Sclerosis. ADHD is multifactorial just like diabetes.

Changes in catecholamine levels and the serotonin transporter may be adaptations to inflammation during development. Norepinephrine, dopamine and serotonin influence inflammation. For example, norepinephrine and dopamine regulate the release of cytokines such as IL-1, IL-6 and TNF alpha from monocytes, macrophages, neutrophils, lymphocytes and endothelial cells. Some cytokines inhibit inflammation but when defective may lead to increased inflammation. It is believed that defective or improperly regulated cytokines induce inflammation and this may lead to multiple diseases including ADHD.

It is likely that ADHD individuals inherit a genetic predisposition to ADHD such as inheriting a defect in SLC6A4. The particular genetic predisposition associated with ADHD is thought to lead to adaptations that likely include inflammation and abnormalities in the endocrine system. It is well established that the immune system, neurotransmitters and endocrine system all influence each other. The second messenger systems are impacted by defects in these systems and vice versa.

Asthma is associated with ADHD 75% of the time. Fibromyalgia is 40-60%. PMS is about 40-60% and it has been noted that many females having ADHD have premature ovarian failure or hysterectomies secondary to other abnormalities such as endometriosis. It has also been noted that a lot of men having ADHD have low levels of testosterone and the rate of breast and prostate cancer in families and patients is elevated along with autoimmune diseases. Medical conditions that occur on one side of the family often aid in identifying the parent that has ADHD when treating a child.

Molecular genetic studies have revealed a possible mechanism by which ADHD may be associated with the inflammatory response. The gene for IL-1 receptor A is found on chromosome 2, in region q12. In a study involving 86 children, it was found that children who had 2 copies of this receptor gene had a lower incidence of ADHD, while children with 4 copies met the criteria for ADHD. In addition, genes for IL-18 receptor 1 and IL-18 accessory protein are found next to the IL-1 receptor gene. IL-18 has been linked with Multiple Sclerosis (MS) and Systemic Lupus Erythematosus (SLE), and ADHD may be coincidental with MS. In some instances, treatment of ADHD/MS patients with SSRIs has worsened MS symptoms, perhaps by lowering dopamine via the multiple autoreceptors for serotonin such as 5HT2. However, treating the ADHD component in such ADHD/MS patients improved their MS symptoms. Additionally, patients have had pain with fibromyalgia and PMS disappear, migraines, allergies and asthma improve. Blood pressures commonly decrease in adults by 10-20 points and rarely up to 50 mmHg or more. Finally, patients have been able to eliminate cholesterol lowering medications and occasionally, if early in treatment, cease taking glucophage or thyroid medication.

Treatment and/or Prevention of Diseases Associated with Inflammation

Inflammation is increasingly regarded as a primary factor in the development of multiple chronic diseases. The inflammatory response provides protection against certain infectious agents including microorganisms, and in this respect is protective of the body. However, left unchecked, inflammation can have adverse effects on virtually every organ system in the body. Chronic inflammation is believed to underlie the development of many chronic diseases including cancer, heart disease, and diseases of the brain, and immune system.

As such, a growing challenge for medical science is to devise better treatments and/or preventive measures for reducing or eliminating chronic inflammation as a means to treat or prevent diseases associated with inflammation. The present invention solves this problem by administering one or more CNS stimulant(s) at a therapeutically effective dosage such that effective serum levels of stimulant(s) are maintained substantially around the clock. For example, in one embodiment a CNS stimulant is administered to a patient having inflammatory disease one or more times per day, preferably one time per day, to achieve a steady state therapeutically-effective serum level that is maintained substantially around the clock. For example, a patient may be administered a sustained-release formulation that delivers stimulant over about 24 and ½ hours to about 36 hours, preferably for about 24 and ½ hours to about 26-27 hours or longer, and most preferable for about 24 and ½ hours to about 25-27 hours or longer following administration.

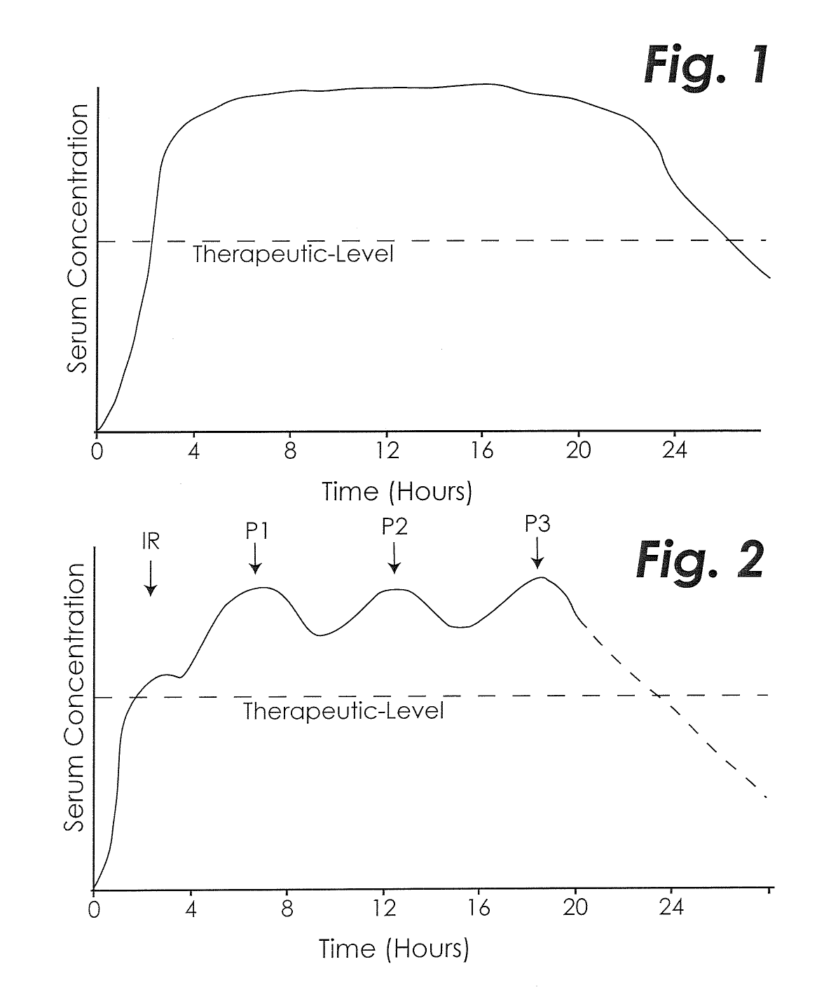

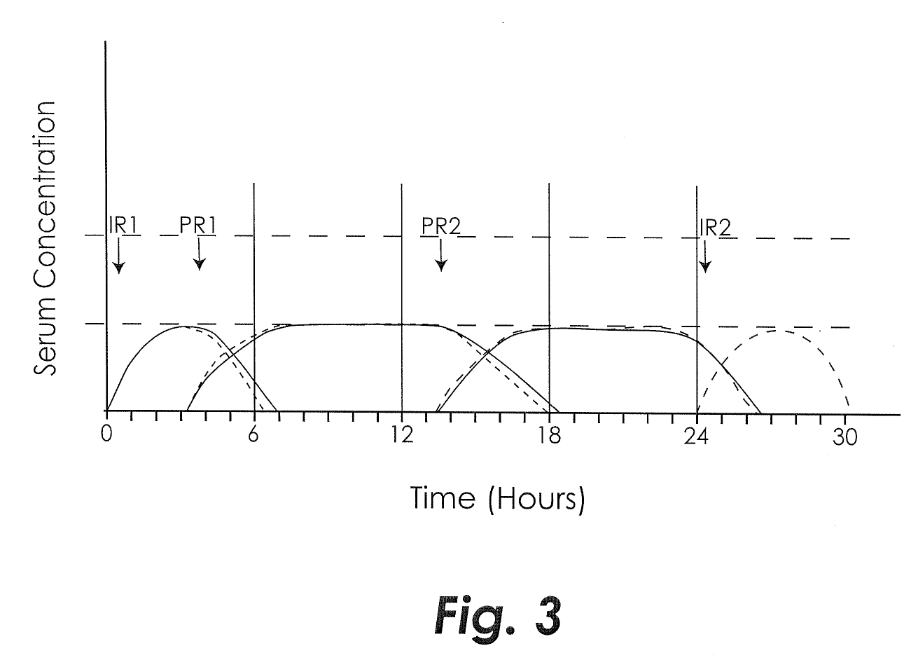

FIGS. 1 and 2 display one embodiment of an idealized target serum profiles for a stimulant administered according to this illustrative embodiment of the invention. FIG. 1 represents a square-wave serum profile in which an initial immediate release of drug is followed by one or more delayed releases that maintain a plateau steady state level within a therapeutically effective range substantially around the clock. FIG. 2 shows another idealized embodiment in which an idealized pulsed-release profile provides an immediate release of drug followed by from one to two, three, four, five or more delayed release pulses that maintain a steady state within a therapeutically effective window substantially around the clock.

A number of CNS stimulants and marketed products are suitable for use according to the present method. For example, suitable CNS stimulants include, but are not limited to: immediate release Methylphenidate products (marketed as Ritalin® 5 mg, 10 mg, 20 mg tablets, Focalin® 2.5, 5, 10 mg tablet, or Methylin®) 5, 10, 20 mg tablet; immediate release mixed amphetamine salts (Dextroamphetamine/Levoampetamine) including Adderall® 5, 10, 20, 30 mg tablet; immediate release Dextroamphetamine including Dexedrine® 5 mg tablet and Dextrostat® 5 and 10 mg tablet. These and other immediate-release stimulant products typically have a duration of about 3-6 hours per dose.

Administering an immediate-release product typically would require 4 to 8 dosings per 24 hour period; preferably from 4 to 6 dosings per 24 hour period. Preferably, CNS stimulant products provide sustained-release of a stimulant, for example, products such as Ritalin SR® 20 mg tablet, Ritalin LA® 10, 20, 30, 40 mg capsule, Focalin® XR 5, 10, 15, 20 mg, Metadate ER® 10, 20 mg tablet, Methylin ER® 10, 20, 40 mg tablet, Metadate CD® 10, 20, 30 mg capsule, and Concerta® 18, 27, 36, and 54 mg capsule; Dexedrine Spansule® 5, 10, 15 mg; and Adderall XR® 5, 10, 15, 20, 25, 30 mg capsule. Other sustained release products include administration by transdermal patch, for example, Daytrana™ (methylphenidate 10 mg, 15 mg, 20 mg, or 30 mg) applied once per day for 9 hours.

Suitable compounds and commercially available products include Amphetamines such as Dextroamphetamine, available in a regular formulation as Dexedrine, having a duration of 4-6 hours per dose. Dexedrine can be administered 3 to 5 times daily. Dexedrine Spansule® provides a long-acting formulation having a duration of about 8-12 hours per dose. Dexedrine Spansule® can be administered according to the present method twice a day, but the duration and release system is not predictable. Adderall® is a mixture of dextroamphetamine and levoamphetamine salts. Adderall® is available in a regular formulation, having a duration of 4-6 hours a dose. Adderall® XR provides a long-acting formulation with a duration of 7-12 hours. Adderall® XR may be administered two to three times a day. Methamphetamine is available in a regular formulation, sold as Desoxyn®.

A patient may be given a product such as Adderall® XR, Focalin® XR, or Ritalin® LA, most preferably Focalin® XR or Adderall® XR, two to three times a day.

Alternatively, if the medication lasts more than 8 hours, twice daily dosing would be inappropriate. However, while Concerta® generally would be given twice a day, it delivers unequal levels that are not optimized for treatment. The plasma level of methylphenidate is low in the morning and is higher in the afternoon. If a patient does well in the morning they may do poorly in the afternoon and their vital signs may go up. The release of stimulant from Concerta® does not mimic what is physiologically normal. Daytrana also delivers increasing amounts of medication which peaks around 9-10 hours. People often struggle in the morning when the level is low. They may be overstimulated 9-10 hours later and experience side effects. Vyvanse does not deliver an even level of medication either.

A therapeutically-effective dosage of stimulant for this aspect of the invention will depend on a number of factors including the type and severity of the disease and/or chronic inflammation, general health, family health, history, age, sex, bodyweight, absorption, metabolism and genetic form of the disease. The skilled practitioner will be able to determine the most appropriate dosage based on these and other factors. Generally, a therapeutically-effective dose would be determined by titrating increasing doses over a period of several days or weeks, with careful monitoring of a patient's response including vital signs (heart rate and blood pressure), ability to nap or sleep while the medication is present, and general feeling or performance on defined tasks. It is desired to find a dosage that normalizes catecholamine levels without under-stimulating or over-stimulating the patient. If a patient's vital signs are high and/or a patient is not able to sleep normally or is not feeling better or performing better in school or on the job, then a lower dosage may be more appropriate. For example, a patient desirably would have vital signs taken prior to beginning medication and then at regular intervals after starting medication and/or after increasing the dose. Vital signs may go up for a day or two by 5 to 10 points but then return to normal. If vital signs do not drop and/or the patient is not able to sleep, the dose is likely too high. For example, with Adderall® XR a patient might be started on 5 mg twice or three times a day. Thereafter, the dose may be increased at weekly intervals in increments of 5 mgs to find an appropriate dose. For methylphenidate products such as Ritalin® SR or Ritalin® LA, a patient could be started, for example, on 10 mg twice or three times a day. Thereafter the dose may be increased at weekly intervals, for example in increments of 10 mg, to find an appropriate dose. The dose should be increased at the lowest mg amount available. The patient should always feel better, perform better and be more efficient and experience benefit. If a patient feels worse or performs at a lower level, this could indicate that the wrong medication or wrong dosing has been provided or other factors may be involved such as low testosterone levels.

Generally, depending on the dosage form, individual patient, and formulation, a therapeutically-effective dosage of methylphenidate is expected to be in the range of 5 to 400 mg per 24 hour period; preferably between 10 to 300 mg/24 hours; more preferably 15 to 250 mg/24 hours; more preferably still between 20 to 100 mg/24 hours; and most preferably, 20 to 60 mg/24 hours. For amphetamine based products such as Adderall®, a therapeutically-effective dosage would generally be expected to be in the range of 5 to 100 mg of a stimulant administered per 24 hour period; preferably between 10 to 80 mg/24 hours; more preferably 20 to 60 mg/24 hours; more preferably still between 30 to 50 mg/24 hours; and most preferably, 40 to 50 mg/24 hours. However, a therapeutically-effective amount given to a patient in a 24 hour period is expected to vary from individual to individual. It is desired to administer an amount of medication to a patient to maintain a normal level of catecholamines at a steady state substantially around the clock.

Desirable serum levels of stimulant will vary depending on the particular stimulant and metabolic characteristics of individual patients. Generally, for methylphenidate products such as Ritalin® or Ritalin® SR, for example, a sustained serum level of between about 3 to 8 ng/ml; preferably between about 4 to 7 ng/ml; and most preferably between about 4 to 6 ng/ml is appropriate for a 20 mg dose. For amphetamine based products such as Adderall®, a sustained serum level of between about 5 to 10 ng/ml; preferably between about 5 to 8 ng/ml of 1-amphetamine, and serum levels of between about 10 to 30 ng/ml, preferably from 10 to 20 ng/ml, most preferably from about 10 to 15 ng/ml of d-amphetamine is desired for a 20 mg dose. However, these dosings are exemplary and do not take into account the various genetic factors and other factors impacting the ideal dose for a particular individual. Serum or urine catecholamine levels can be checked to assist in making sure a patient is getting an appropriate dose of medication, depending on their age. In an illustrative embodiment, a CNS stimulant is administered to a patient with chronic inflammation and a history of cancer in the family in a suitable dosage form, for example, as a tablet or capsule, once per day, i.e., one time per 24 hour period. In another embodiment a preferred dosage form of sustained release stimulant further comprises one or more anti-inflammatory agent(s), for example, fish oil, DHA, EPA, GLA, pomegranate extract, NSAID, or grape seed extract. A suitable sustained release dosage form desirably provides an immediate release pulse of stimulant that reaches therapeutically-effective steady state serum levels within about 30 minutes to about 4 hours; preferably between about 30 minutes to about 2 hours after ingestion. Thereafter, a second, optionally, third, fourth, fifth, sixth, or seventh delayed pulse is released. Each delayed-release pulse is released from about 4 hours to 8 hours after the immediately preceding pulse.

Absorption of the particular stimulant administered will depend upon factors such as the acid and base balance in the body, inflammation, and differences in the lining of the gastrointestinal tract. For example, methylphenidate products absorb better in acid environments while amphetamine products absorb better in basic environments.

In one embodiment, a once-daily dosage form provides an immediate release of stimulant that reaches therapeutically effective steady state serum levels within about 30 minutes to about 2-4 hours, more preferably from about 30 minutes to about 2-3 hours, and thereafter provides a sustained release of stimulant at steady state, therapeutically effective levels substantially around the clock, for example, for from about 24 and ½ to about 36 hours, preferably from about 24 and ½ to about 26-27 hours or more, and most preferably from about 24 and ½ to about 25-27 hours or longer after administration. FIG. 1 illustrates a target serum profile that could be achieved by any number of well-known pharmaceutical formulation techniques for producing time-targeted, pH-dependent, or pH independent drug release, for example, gastric or enteric release, or otherwise comprising sustained release drug products. Suitable delayed-release techniques known to the skilled artisan include use of spheres, beads, pellets, powders, matrix materials that comprise or are coated with active ingredient and which typically comprise or are coated with additional layering to delay release of an active agent. A number of well known compounds, compositions and techniques are known to the skilled artisan for producing a desired delayed release profile for the active agent including, for example, various acrylic polymers such as Eudragit® (Rohm Pharma), hydrophobic materials such as alkylcellulose (e.g. Aquacoat®, FMC Corp. Philadelphia, Pa.), plasticizer materials, and matrix materials such as gums, alkylcelluloses, cellulose ethers, and acrylic resins such as acrylic polymers and copolymers. Suitable methods and reagents are described in U.S. Pat. No. 7,083,808, the entire contents of which is incorporated herein by reference.

In another embodiment, a CNS stimulant is administered to an individual as a tablet or capsule, more than once per day, for example, two, three, or four times per 24 hour period. A suitable sustained release dosage form for more than one time per day dosing desirably provides an immediate release pulse of stimulant that reaches therapeutically-effective steady state serum levels within about 30 minutes to about 4 hours; preferably between about 30 minutes to about 2-3 hours after ingestion. Thereafter, second and optionally, third, fourth, fifth, sixth, or seventh delayed pulses are released. Each delayed-release pulse is released from about 4 hours to 8 hours after the preceding pulse, preferably from 4 hours to 5 hours after the preceding pulse.

In another embodiment, a stimulant is administered transdermally for about 24 hours, alternatively for a period between 1 day and 3 days; alternatively for a period between 1 day and 7 days; alternatively once per week, twice per week, or once every two weeks. When administered transdermally, a stimulant drug such as methylphenidate, for example, is provided in a skin patch, such that the stimulant is administered in a range of 0.5 mg/24 hours to about 100 mg/24 hours, preferably from about 2.5 to 20 mg/24 hours, in a device containing from about 20 to 180 mg of methylphenidate.

An illustrative embodiment relates to co-administering one or more CNS stimulant(s) plus one or more anti-inflammatory agent(s), together in a single dosage form or separately, e.g. by simultaneous or sequential administration. Suitable anti-inflammatory agents include synthetic as well as natural compounds including, but not limited to NSAIDs, fish oil, DHA, EPA, omega-3 fatty acids and pomegranate juice or extract. In one embodiment, the present method relates to co-administering a CNS stimulant with one or more natural product(s), for example, fish oil, omega-3 fatty acids DHA and EPA (available commercially as OMACOR®, Reliant Pharmaceuticals), or pomegranate juice or extract, grape seed extract, vitamin E, or the sulfated polysaccharide Fucoidan. Appropriate dosages of DHA and EPA are from about 650 mg to 3 grams per day; preferably from 650 mgs to 1 gram per day. Appropriate dosages of pomegranate extract are from about 100 to 500 mg/day; preferably from about 200 to 250 mg/day. Appropriate dosages of grape seed extract are from about 100 to 500 mg/day; more preferably from about 200 to 400 mg/day, most preferably about 300 mg/day.

Another embodiment of the invention relates to administering pyridoxal 5′ phosphate (P-5-P or PLP) alone or in combination with one or more CNS stimulant(s) and/or anti-inflammatory agents. Pyridoxal 5′ phosphate is the active form of vitamin B6, which is a cofactor in more than 100 chemical reactions in the body, in particular reactions pertaining to amino acids and lipid metabolism, hormone levels, neurotransmitter synthesis, and inflammation. Low plasma levels of P-5-P or PLP may inversely correlate with high plasma homocysteine levels and increased risk of coronary artery disease. A growing body of evidence indicates an inverse relationship between plasma P-5-P or PLP levels and inflammation associated with, for example, rheumatoid arthritis, inflammatory bowel disease, and atherosclerosis. (S. Friso et al., Am. J. Clin. Nutr., 79, 992-998, 2004). If co-administered, CNS stimulant(s) and pyridoxal 5′ phosphate (P-5-P or PLP) may be administered together in a single dosage form, or separately. Co-administration may be by any suitable means well known to the skilled artisan including simultaneous administration or sequential administration. It is believed that gastrointestinal inflammation reduces absorption of some amino acids from the gastrointestinal tract which in turn adversely affects maintenance of normal catecholamine levels. When administering P-5-P or PLP a suitable dosage would be in a range of about 50 mg to about 100 mg/day. In some cases 150 mgs a day may be needed. Co-administering P-5-P with a stimulant avoids feedback inhibition by catecholamines on pyridoxal kinase, which has been seen with L-DOPA. This facilitates maintenance of catecholamine levels and other biochemical pathways associated with P-5-P. A majority of ADHD patients may be defective in their ability to synthesize vitamin B6, which impacts their amino acid levels and contributes to other diseases as well. It is important to make sure Thiamin, Riboflavin, Niacin, Pantothenic Acid and Vitamin B12 levels are normal and maintained in a normal range. If too much vitamin B6 is replaced, it is possible that other vitamin B levels may be adversely altered. However, restoring PLP to a physiologically appropriate level may correct other abnormalities in the B vitamins and help maintain correct levels.

The gene encoding serine hydroxlmethyltransferase maps to chromosome 17p11.2, which is the same location as ADHD2. The gene encoding pyridoxine 5 phosphate oxidase is located on chromosome 17q21.32 and manufactures P-5-P and ammonia. This region of chromosome 17 may overlap with SLC6A4 and impact P-5-P or PLP levels if defective. The gene encoding pyridoxyl phosphate phosphatase is located on chromosome 22cen-q12.3 and is in the same region as COMT. Finally, Pyridoxal Kinase is located on 21q22.3. Several other enzymes involved in B6 synthesis have yet to be located and several enzymes may rely on bacteria in the Gastrointestinal system. Chromosome 13 may also be a location for gene(s) encoding one or more enzymes that influence vitamin B6 levels. For example, an ADHD patient with a defect in 5HT2A also has a vitamin B6 level of 100, well over the normal level of 32. The patient also has a defective BRCA2 gene and one of her daughters has ADHD, a high vitamin B6 level and the same amino acid profile. However, it is likely that this is a result of a common feedback pathway or even altered bacteria in her gastrointestinal tract. Thus, vitamin B6 or P-5-P appears to play a major role in ADHD. It is an antioxidant and low levels are now being mentioned as a risk factor for breast cancer. It is important to maintain P-5-P levels and not inhibit pyridoxal kinase while treating a person for ADHD. However, if a person does have dopamine feedback on pyridoxal kinase, P-5-P will still serve as a co-factor for numerous biochemical pathways in the body and maintain catecholamine synthesis; hopefully, lowering the amount of medication needed to return a person to normal physiologic functioning. Additionally, this may help prevent tics and possibly other diseases.

Treatment of ADHD with CNS Stimulants for Extended Periods

It has long been recognized that many ADHD patients respond to treatment with central nervous system (CNS) stimulants, which work by stimulating the areas of the brain responsible for focus, attention, and impulse control. It has also been recognized that use of CNS stimulants to treat ADHD carries certain risks including the risk of substance abuse and undesirable side effects including increased heart rate and blood pressure, decreased appetite, headache, insomnia, psychosis, and irritability. For these and other reasons physicians must carefully monitor a patient's response to insure that the clinical benefits exceed the risks of undesired effects. Dosing on weight alone is dangerous. One amino acid, taurine, is commonly depleted in people with ADHD. Because of its association with cardiac tissue and ion metabolism, low levels of taurine may be accompanied by cardiac arrhythmias and seizures. Overmedication of a patient that is depleted in this amino acid or others that make up cardiac tissue could be harmful. Thus, the typical approach to the treatment of ADHD needs to change. People often have a hard time focusing and suffer from anxiety when they are deficient in this amino acid, but it has numerous functions in the body and even regulates metalloproteins.

Metalloproteins impact the ratios of certain metals in the body such as copper and zinc. When there is a defect in metalloproteins, a person will experience an imbalance in these metals which can impact inflammation and function of essential co-factors. Over time these metals can accumulate and cause diseases. This can impact levels of neurotransmitters, hormones and second messenger systems.

Prior courses of treatment with CNS stimulants involved careful monitoring of patient response, and generally dosing only for limited portions of each day. In particular, stimulant treatments for ADHD targeted treatment periods of from 8 to 12 hours per day, the objective being to improve symptoms during the waking hours. The focus was on improving performance in school or on the job while not impairing a patient's ability to sleep at night. Often, patients were slightly overmedicated, and displayed elevated vital signs. Prior treatment regimens often made patients slightly worse and lead to undesirable side effects like diminished appetites. Significantly, prior treatment regimens did not normalize catecholamine levels throughout the day, let alone substantially around the clock.

It is well known that by age 21, 80% of people with ADHD have trouble with sleep and insomnia secondary to a lack of a normal level of catecholamines at night while a person is sleeping. The methods of the present invention are expected to restore normal sleep patterns by treating the disease appropriately at steady state and around the clock. Additionally, the invention is expected to improve electrolyte balance, acid base balance, endocrine labs, decrease inflammation and pain, improve pulmonary function in people with asthma, and lower blood pressure, along with possibly healing brain structures. If the wrong medication is used or if the proper dose is exceeded, catecholamines begin to activate different receptors and increase inflammation and free radical production all over the body and in the brain. An anti-inflammatory agent and antioxidant offer a buffer in case this occurs. Current stimulant medications do not treat the disease around the clock and through the night. Additionally, they do not maintain a physiologically normal steady state. As a result, people of have difficulty with sleep as they are over or under stimulated.

In determining a correct dose, weight is only one factor. The absorption of stimulant medications can vary by 700% and people metabolize them at different rates. People also have different genetic defects causing this disease and that changes the amount of medication needed. A 24 hour medicine at the correct dose is needed to make a person normal and prevent and treat disease. For example, treatment of one four year old female who was about to be put on growth hormone led to her growing 5.8 inches in several months and an increase in her growth hormone. This understanding may also explain why one study indicates that six cups of coffee a day (which works through adenosine 2 receptor antagonism to increase dopamine and norepinephrine) prevents the onset of Parkinson's disease. Cigarettes or nicotine may also protect family members with a history of Parkinson's disease in their family. Both are stimulants. However, neither caffeine nor nicotine are nearly as effective at treating ADHD and both have unwanted side effects.

Diabetes is treated 24 hours a day and, as a genetic disease, ADHD needs to be treated the same. Additionally, there is only one correct amount of insulin to give a person with diabetes and everyone with diabetes requires fine tuning the medication. Similarly, the dose of medication for ADHD should be fine tuned. Weight is only one factor to consider in finding an appropriate, therapeutically-effective dose. It is desired to restore catecholamines to an even, normal level. It is also desirable to correct other abnormalities that are associated with ADHD to prevent or reduce the risk of future diseases. For example, if a patient lacks sufficient arginine, and has a high vitamin B6 level, P-5-P may be given, optionally also giving arginine on a temporary basis. It is expected that such a patient will now release growth hormone, eliminate nitrogen from the body, lose weight and decrease their blood pressure and have better insulin secretion. This also lowers IL-6 and helps prevent tumors. ADHD is not so simple and such complexity is frequently clinically observed.

The illustrative embodiments of the present invention have surprisingly found that when ADHD patients are treated with a therapeutically-effective dosage of stimulants for periods longer than previously prescribed methods, i.e. for periods substantially around the clock, patients feel better, sleep better, and perform better at school or on the job. It is believed that an extended treatment period benefits the patient by leading to long-term normalized catecholamine levels, reduced inflammation and a normalized endocrine system. Additionally, catecholamines release BDNF, which stimulates new brain cell growth and increases dendritic connections. When catecholamine levels are normalized substantially around the clock, patients recover a normal sleep cycle and are able to turn their minds off. People with ADHD have been shown to have theta waves in the morning instead of alpha waves on EEGs. When ADHD is treated during the night with an appropriate amount of stimulant, cortisol release is inhibited as normal sleep occurs. When sleep is disrupted, the body releases cortisol to elevate blood sugar which over long periods can lead to weight gain, insulin resistance and aging. With normal sleep, the body releases Growth hormone (GH) in the morning which promotes antioxidant activity and maintains muscle mass, health and prevents aging. Additionally, norepinephrine is required to release melatonin, which is an antioxidant and restores normal sleep. Dopamine inhibits periodic leg movements and catecholamines help maintain air flow. Treating a genetic disease 8 hours out of 24 hours will fail. If diabetes were treated this way, what could be prevented would never be realized. This approach also allows depression to be treated.

More specifically, if ADHD is treated substantially around the clock, the medial prefrontal cortex and the dorsolateral prefrontal cortex (DLPFC) may regenerate. It is also possible that the striatum and the vermis of the cerebellum will be repaired. The dorsolateral prefrontal cortex is involved in learning, cognition, judgment, abstraction, and reasoning, and continues to develop into late adolescence. This part of the brain is thought to be intimately involved in ADHD. The medial prefrontal cortex and the dorsolateral prefrontal cortex have demonstrated plasticity and are affected by different concentrations of BDNF, a protein that has the capacity to increase nerve projections and dendritic connections in various disease states. These particular areas of the brain have been shown to respond to treatments that increase BDNF. For example, experimental evidence in a rat model of ADHD has shown that after 12 days of treatment with continuous administration of amphetamine, rats showed an increase in dendritic length and branches of the pyramidal neurons in the medial prefrontal cortex. The latter structure in the rat brain serves some of the same functions as the dorsolateral prefrontal cortex in humans.

It is known that norepinephrine and dopamine stimulate release of BDNF and decrease glutamate in the human brain, which results in new brain cell growth, and reduced cell death. Norepinephrine, at the correct dose, releases BDNF in the frontal cortex and decreases abnormal levels of glutamate. Dopamine does the same in the striatum. Interestingly, fish oils increase BDNF in the brain and activate BCL-2 which helps repair gray matter. In addition, fish oil lowers proinflammatory cytokines and reduces damage by glutamate to the brain. Fish oil rich in DHA and EPA, also reduces pro-inflammatory cytokines IL-1, IL-6, and TNF alpha, and stops glutamate release in the brain. DHA is a major part of cell membranes in the Central Nervous system and keeps membranes healthy and fluid. Dopamine also lowers IL-1, IL-6, and TNF alpha and lowers glutamate. Norepinephrine will stop the glutamate damage to the frontal lobes and release BDNF. By restoring catecholamine levels substantially around the clock the present method may help reduce or prevent damage to the brain.

A number of CNS stimulant compounds and marketed products are suitable for use according to the present method. For example, suitable CNS stimulants include, but are not limited to: immediate release Methylphenidate products (marketed as Ritalin® 5 mg, 10 mg, 20 mg tablets, Focalin® 2.5, 5, 10 mg tablet, or Methylin®) 5, 10, 20 mg tablet; immediate release mixed amphetamine salts (Dextroamphetamine/Levoampetamine) including Adderall® 5, 10, 20, 30 mg tablet; immediate release Dextroamphetamine including Dexedrine® 5 mg tablet and Dextrostat® 5 and 10 mg tablet. These and other immediate-release stimulant products typically have a duration of about 3-6 hours per dose.

Administering an immediate-release product typically would require 4 to 8 dosings per 24 hour period; preferably from 4 to 6 dosings per 24 hour period. Preferably, CNS stimulant products provide sustained-release of a stimulant, for example, products such as Ritalin SR® 20 mg tablet, Ritalin LA® 10, 20, 30, 40 mg capsule, Focalin® XR 5, 10, 15, 20 mg, Metadate ER® 10, 20 mg tablet, Methylin ER® 10, 20 mg tablet, Metadate CD® 10, 20, 30 mg capsule, and Concerta® 18, 27, 36, and 54 mg capsule; Dexedrine Spansule® 5, 10, 15 mg; and Adderall XR® 5, 10, 15 20, 25, 30 mg capsule. Other sustained release products include administration by transdermal patch, for example, Daytrana™ (methylphenidate 10 mg, 15 mg, 20 mg, or 30 mg) applied once per day. Administering a sustained-release product would generally involve providing from 2 to 3 dosings to a patient per day, depending on the particular patient and the particular product. In any case it is desirable to administer a stimulant such that therapeutically effective amounts are present substantially around the clock, for example, from about 24½ to about 36 hours, more preferably for about 24 and ½ to about 26-27 hours or longer, most preferably from about 24½ to about 25-27 hours per day or longer if needed to maintain a steady state while the next dose of medication is absorbing.

Suitable compounds and commercially available products include Amphetamines such as Dextroamphetamine, available in a regular formulation as Dexedrine, having a duration of 4-6 hours per dose. Dexedrine can be administered 3 to 5 times daily. Dexedrine Spansule® provides a long-acting formulation having a duration of about 6-12 hours per dose. Dexedrine Spansule® can be administered according to the present method two to three times a day. Adderall® is a mixture of dextroamphetamine and laevoamphetamine salts. Adderall® is available in a regular formulation, having a duration of 4-6 hours a dose. Adderall® XR provides a long-acting formulation with a duration of 7 to 12 hours. Adderall® XR may be administered two to three times a day. Methamphetamine is available in a regular formulation, sold as Desoxyn®, by Ovation Pharmaceutical Company.

A patient treated may be given a product such as Adderall® XR, Focalin® XR, or Ritalin® LA, most preferably Focalin® XR or Adderall® XR, two to three times a day. Data was collected on Adderall XR® dosed once a day for 24 months and the heart rate or blood pressure did not change at the appropriate dose. It has been demonstrated that stimulants decrease the rate of sudden death due to cardiac events from 3.3 out of 100,000 to 0.6 out of 100,000 in adolescents on the appropriate dose of medication.

A therapeutically-effective dosage of stimulant to achieve the desired therapeutic benefit according to the present invention will depend on a number of factors including the type and severity of the disease, general health, age, sex, bodyweight, absorption, metabolism and genetic cause of the disease to name a few. The objective is to find the correct medication and the correct dose that returns a person to normal functioning and physiology. The skilled artisan will be able to determine a therapeutically-effective dosage based on these and other factors. Generally, a therapeutically-effective dose would be determined by titrating increasing doses over a period of several days or weeks, with careful monitoring of a patient's response including vital signs (heart rate and blood pressure), ability to nap or sleep, and general feeling or performance on defined tasks. Determining an optimal therapeutically-effective dose for a given patient may also be monitored by the measurement of neurotransmitter levels. It is desired to find a dosage that normalizes catecholamine levels without under-stimulating or over-stimulating the patient. If a patient's vital signs are high and/or a patient is not able to sleep normally or is not feeling better or performing better in school or on the job, then a lower dosage may be more appropriate. Additionally, they may respond better to a different stimulant. For example, a patient should have vital signs taken prior to beginning medication and then at regular intervals after starting medication and/or after increasing the dose. Vital signs may go up for a day or two by 5 to 10 points but then return to normal. If vital signs do not drop and/or the patient is not able to sleep or perform well, the dose is likely too high. For example, with Adderall® XR a patient might be started on 5 mg twice or three times a day. Thereafter, the dose may be increased at weekly intervals in increments of 5 mgs to find an appropriate dose. For methylphenidate products such as Ritalin® SR or Ritalin® LA, a patient could be started, for example, on 10 mg twice or three times a day. Thereafter the dose may be increased at weekly intervals, for example in increments of 10 mg, to find an appropriate dose. The dose should be increased at the lowest mg amount available in order to fine tune the dose to make a person physiologically normal.

Generally, depending on the dosage form and formulation, a dosage of methylphenidate would be administered in the range of 5 to 400 mg per 24 hour period; preferably between 10 to 300 mg/24 hours; more preferably 15 to 250 mg/24 hours; more preferably still between 20 to 100 mg/24 hours; and most preferably, 20 to 60 mg/24 hours. For amphetamine based products such as Adderall®, a dosage in the range of 5 to 100 mg of a stimulant will be administered per 24 hour period; preferably between 10 to 80 mg/24 hours; more preferably 20 to 60 mg/24 hours; more preferably still between 30 to 50 mg/24 hours; and most preferably, 40 to 50 mg/24 hours.

Desirable serum levels of stimulant will vary depending on the particular stimulant and metabolic characteristics of individual patients. Generally, for methylphenidate products such as Ritalin® or Ritalin® SR, for example, a sustained serum level of between about 3 to 8 ng/ml; preferably between about 4 to 7 ng/ml; and most preferably between about 4 to 6 ng/ml is appropriate for a 20 mg dose. For amphetamine based products such as Adderall®, a sustained serum level of between about 5 to 10 ng/ml; preferably between about 5 to 8 ng/ml of 1-amphetamine, and serum levels of between about 10 to 30 ng/ml, preferably from 10 to 20 ng/ml, most preferably from about 10 to 15 ng/ml of d-amphetamine is desired for a 20 mg dose. It is to be understood that these are averages and individual patients may fall outside these ranges. Genetics and other factors will change the therapeutic amounts for individual patients. Every patient will be different and must be treated that way. Patients will have different levels of neurotransmitters and varying defects in amino acids, hormones, and inflammation that all impact what will be a therapeutically-effective dose.

In an illustrative embodiment, a CNS stimulant is administered to an ADHD patient in a suitable oral dosage form, for example, as a tablet or capsule, preferably once per day, i.e. one time per 24 hour period to maintain a steady state, therapeutically effective level of drug substantially around the clock. In another embodiment a dosage form of sustained release stimulant further comprises one or more anti-inflammatory agent(s), for example, fish oil, DHA, EPA, pomegranate extract, NSAID, or grape seed extract. In another embodiment of the present invention, a preferred dosage form of sustained release stimulant(s) further comprises pyridoxal 5′ phosphate (P-5-P or PLP). In another embodiment, a dosage form of the present invention comprises a sustained release formulation of CNS stimulant(s), one or more anti-inflammatory agent(s), as described above, and pyridoxal 5′ phosphate to maintain basic biochemical pathways and even increase their activity. A suitable sustained release dosage form desirably provides an immediate release pulse of stimulant that reaches therapeutically-effective serum levels within about 30 minutes to about 4 hours; preferably between about 30 minutes to about 2-3 hours after ingestion. Thereafter, a second, optionally, third, fourth, fifth, sixth, or seventh delayed pulse is released. Each delayed-release pulse is released from about 4 hours to 6 hours after the preceding pulse, preferably from 4 hours to 5 hours after the preceding pulse. An immediate pulse allows a fast onset of action while later pulses maintain a steady state and then terminate gradually while the next dose of medication is absorbed and increasing in plasma concentration to maintain a steady state substantially around the clock. If a dosage form contains other agents such as P-5-P or PLP and/or anti-inflammatory agent(s), release of said agents may be substantially immediate after administration or as a delayed or sustained release.

In one embodiment, a once-daily dosage form provides an immediate release of stimulant that reaches therapeutically effective serum levels within about 30 minutes to about 4 hours, more preferably from about 30 minutes to about 2-3 hours, and then provides a sustained release of stimulant to achieve steady state, therapeutically effective levels substantially around the clock. FIG. 1 illustrates a target serum profile that could be achieved by any number of well-known pharmaceutical formulation techniques for producing time-targeted, pH-dependent, or pH independent delayed-release, for example, gastric or enteric release, or otherwise comprising sustained release drug products. Such techniques include use of spheres, beads, pellets, powders, matrix materials that comprise or are coated with active ingredient and which typically comprise or are coated with additional layering to delay release of active agent.

In another embodiment, a CNS stimulant is administered to an ADHD patient as a tablet or capsule, more than once per day, for example, two, three, or four times per 24 hour period. A suitable sustained release dosage form for more than one time per day dosing desirably provides an immediate release pulse of stimulant that reaches therapeutically-effective serum levels within about 30 minutes to about 4 hours; preferably between about 30 minutes to about 2-3 hours after ingestion. Thereafter, second, optionally, third, fourth, fifth, sixth, or seventh delayed pulses are released. Each delayed-release pulse is released from about 4 hours to 8 hours after the preceding pulse, preferably from 4 hours to 5 hours after the preceding pulse. In one embodiment, an initial fast pulse is followed by 2 more pulses that release at a slower rate and with a longer interval between release of the medication to achieve steady state and then maintain a steady state substantially around the clock so that when the medication has dropped below therapeutically-effective levels and is being eliminated, the next dose is beginning to be absorbed and take effect. Maintenance of a steady state of medication at an appropriate dose substantially around the clock is the objective.

In another embodiment, a stimulant is administered transdermally for about 24 hours, alternatively for a period between 1 day and 3 days; alternatively for a period between 1 day and 7 days; alternatively once per week, twice per week, or once every two weeks. When administered transdermally, a stimulant drug such as methylphenidate, for example, is provided in a skin patch, such that the stimulant is administered in a range of 0.5 mg/24 hours to about 100 mg/24 hours, preferably from about 2.5 to 20 mg/24 hours, in a device containing from about 20 to 180 mg of methylphenidate. Alternatively, a stimulant may be administered by a pump or other suitable drug delivery device such as the Azlet pump available from Alza Corporation.

An illustrative embodiment, relates to co-administering one or more CNS stimulant(s) plus one or more anti-inflammatory agent(s), together in a single dosage form or separately. Suitable anti-inflammatory agents include synthetic as well as natural compounds including, but not limited to NSAIDs, fish oil, DHA, EPA, omega-3 fatty acids and pomegranate juice or extract. In one embodiment, the present method relates to co-administering a CNS stimulant with one or more natural product(s), for example, fish oil, omega-3 fatty acids DHA and EPA (available commercially as OMACOR®, Reliant Pharmaceuticals), or pomegranate juice or extract, grape seed extract, vitamin E, or the sulfated polysaccharide Fucoidan. Appropriate dosages of DHA and EPA are from about 650 mg to 2 grams per day; preferably from 650 mgs to 1 gram per day. Appropriate dosages of pomegranate extract are from about 100 to 500 mg/day; preferably from about 200 to 250 mg/day. Appropriate dosages of grape seed extract are from about 100 to 500 mg/day; more preferably from about 200 to 400 mg/day, most preferably about 300 mg/day.

Another embodiment of the invention relates to co-administering one or more CNS stimulant(s) with pyridoxal 5′ phosphate (P-5-P or PLP), together in a single dosage form, or separately as a treatment for ADHD. Co-administration may be by any suitable means well known to the skilled artisan including simultaneous administration or sequential administration. Pyridoxal 5′ phosphate (available commercially as a vitamin supplement) is the active form of vitamin B6. It is believed that ADHD patients are deficient in producing the active form of vitamin B6 due in part at least to gastrointestinal inflammation. It is likely that genetic defects as pointed out are responsible for defects in vitamin B6 synthesis. It is also possible that treatment of ADHD causes feedback and inhibits pyridoxal kinase and the production of P-5-P, interfering with multiple biochemical pathways. Defects in vitamin B6 synthesis likely leads to low levels of P-5-P or PLP and does not allow enzymes throughout the body to function normally. This includes manufacturing catecholamines, serotonin, GABA, melatonin, amino acids, hormones and fatty acids. This abnormal condition will result in inflammation This type of inflammation may alter GI flora, reduce absorption of amino acids, vitamins and nutrients from the gastrointestinal tract, which in turn adversely affects maintenance of normal catecholamine levels too. If P-5-P is maintained at normal levels, people will have enough to manufacture catecholamines, hormones, and lipids properly. Inflammation will not result secondary to feedback inhibition, shutting down numerous important pathways in the body. Suitable dosages for P-5-P or PLP are in a range of about 25-50 mg to about 100 mg per day. In some cases 150 mgs may be needed depending on the defect in the pathway, size and sex of the patient.

Another embodiment of the invention relates to administering pyridoxal 5′ phosphate (P-5-P or PLP) alone or in combination with one or more anti-inflammatory agent(s) as a treatment for ADHD. When co-administered pyridoxal 5′ phosphate (P-5-P or PLP) and an anti-inflammatory agent(s) are administered together in a single dosage form, or separately. Co-administration may be by any suitable means well known to the skilled artisan including simultaneous administration or sequential administration. Suitable anti-inflammatory agents include synthetic as well as natural compounds including, but not limited to NSAIDs, fish oil, DHA, EPA, omega-3 fatty acids and pomegranate juice or extract. In one embodiment, the present method relates to co-administering a CNS stimulant with one or more natural product(s), for example, fish oil, omega-3 fatty acids DHA and EPA (available commercially as OMACOR®, Reliant Pharmaceuticals), or pomegranate juice or extract, grape seed extract, vitamin E, or the sulfated polysaccharide Fucoidan. Appropriate dosages of DHA and EPA are from about 650 mg to 2 grams per day; preferably from 650 to 1 gram per day. Appropriate dosages of pomegranate extract are from about 100 to 500 mg/day; preferably from about 200 to 250 mg/day. Appropriate dosages of grape seed extract are from about 100 to 500 mg/day; more preferably from about 200 to 400 mg/day, most preferably about 300 mg/day. Pyridoxal 5′ phosphate (available commercially as a vitamin supplement) is the active form of vitamin B6. It is believed that ADHD patients may be deficient in producing the active faun of vitamin B6 due in part at least to gastrointestinal inflammation and genetic defects in its synthesis. This type of inflammation reduces absorption of amino acids, nutrients and vitamins from the gastrointestinal tract which in turn adversely affects maintenance of normal catecholamine levels along with a deficit of P-5-P. Suitable dosages for P-5-P or PLP are in a range of about 25-50 mg to about 100 mg per day. In some cases about 150 mgs may be necessary depending on the defect, sex and weight of the individual. The genetics have been outlined herein above. P-5-P functions as a safeguard and prevents feedback inhibition, keeping multiple biochemical pathways functioning.

### Administering Stimulants

CNS stimulants are generally administered orally or, topically (for example, by transdermal patch) to achieve long-acting therapeutically effective treatment.

One embodiment is directed to an oral dosage faun comprising an effective amount of a CNS stimulant including but not limited to methylphenidate, amphetamine, or a pharmaceutically acceptable salt thereof and at least one release modifying material which causes the formulation to provide in-vitro dissolution of the drug of from about 0 to about 45% released after 0.25 hour; from about 10 to about 50% released after about 1 hour; from about 30 to about 80% released after about 4 hours. The oral dosage form when orally administered to a human patient further provides a time to maximum plasma concentration at about 0.5 to about 4 hours after oral administration, preferably at about 0.5 to about 2-3 hours after administration, and a duration of effect which lasts substantially around the clock, wherein most preferably the plasma concentration of the drug rapidly falls about 24 hours after oral administration to below therapeutically-effective levels. In one embodiment, the drug may still be present longer than about 27 hours after administration while a second administration of the drug is being absorbed and achieving steady state or maintaining close to one steady plasma level. In certain illustrative embodiments, the oral dosage form, when orally administered to a patient, provides a peak plasma concentration from about 4 ng/ml to about 6.5 ng/ml per 20 mg dose of methylphenidate contained in the oral dosage form. In certain illustrative embodiments, the oral dosage form, when orally administered, provides a peak plasma concentration from about 5 ng/ml to about 6.5 ng/ml per 20 mg dose of methylphenidate contained in the oral dosage form. In certain further illustrative embodiments, the oral dosage form provides peak plasma concentration from about 1.0 to about 2.0 times the plasma concentration of methylphenidate provided by the formulation at about 24 and ½ to about 25-27 hours or slightly longer after oral administration, and more preferably from about 1.0 to about 1.6 times the plasma concentration of methylphenidate provided by the formulation at about 24 hours after oral administration.

In one embodiment, plasma levels of a stimulant achieve and maintain a square-wave profile substantially around the clock. In one illustrative embodiment, a formulation provides bimodal release and/or biphasic absorption to provide a “plateau” at therapeutically effective levels which lasts substantially around the clock. For example, therapeutically effective plasma levels of stimulant may be present from about 24 and ½ hours to about 36 hours, preferably from about 24 and ½ to about 26-27 hours or slightly longer; most preferably from about 24 and ½ to about 25-27 hours or slightly longer. A second and/or subsequent dose(s) may be administered to achieve and/or maintain a steady state plasma level of the drug and catecholamines substantially around the clock. An immediate-release component preferably represents from about 5% to about 40% of the total dose, more preferably from about 10 to about 25% of total dose, and the controlled release component preferably represents from about 95% to about 60% of the total dose, more preferably from about 90% to about 75% of methylphenidate contained in the formulations. When administering methylphenidate, it is desired that the onset of action occurs from about 0.5 to about 4 hours, and most preferably from about 0.5 to about 2-3 hours after oral administration. It is further desired that the dosage form provides below-effective plasma levels of methylphenidate from about 24 and ½ to about 26-27 hours or longer, more preferably from about 24 and ½ to about 25-27 hours longer, after oral administration. The duration of action may be extended slightly to allow steady state to be maintained substantially around the clock. In a preferred embodiment, a single dose can be administered in the morning before school or work begins to provide beneficial action substantially throughout the succeeding 24 hour period following administration.

In one embodiment, a dosage form is based on controlled-release dosage forms such as, for example, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), or DUREDAS (Dual Release Drug Absorption System), available from Elan Corporation, Dublin, Ireland. A dosage form of an illustrative embodiment is based on SODAS. SODAS relies on the production of uniform spherical beads of 1-2 mm in diameter containing drug plus excipients and coated with controlled-release polymers. Each bead may be coated with stimulant, followed by a number of layers or coatings of an appropriate mix of controlled-release polymers. These polymers (water soluble and insoluble, pH dependent/independent etc.) form a rate-controlling release membrane around each bead. By eliminating a rate control layer drug is released immediately. Preferably, a SODAS or other suitable dosage form provides from 1 to 6 release times to cover 24 hours a day at steady state. Most preferably, a dosage faun provides for once per day dosing to increase patient compliance and convenience. SODAS technology provides a means to achieve the desired serum profile. In particular a SODAS dosage form can be comprised of an immediate release of stimulant followed by sustained releases, which thereafter maintain a steady plasma level substantially around the clock, for example, for about 24 and ½ to about 36 hours, preferably from about 24 and ½ to about 26-27 hours or longer, most preferably for about 24 and ½ to about 25-27 hours or longer after administration. An alternative embodiment, relates to a pulsatile-release dosage form in which a once daily dosage form such as SODAS releases stimulant in multiple bursts throughout the day. Preferably, one release will be immediate to achieve steady state rapidly, while later releases are at a slower rate and at a longer duration between releases to maintain steady state substantially around the clock and allow the drug to decline in concentration while the next dose is ascending in its plasma concentration.

In another embodiment, a dosage form of the present invention provides for once daily tablet or capsule administration in which an immediate release of stimulant within about 30 minutes to about 2-4 hours, preferably from about 30 minutes to about 2-3 hours, is followed by a delayed release component of the dosage form which is sustained at steady state, therapeutically effective levels substantially around the clock, for example, for from about 24 and ½ to about 36 hours after administration, preferably from about 24 and ½ to about 26-27 hours or longer, most preferably from about 24 and ½ to about 25-27 hours after administration or longer to allow the next dose to achieve or maintain steady state substantially around the clock. The skilled artisan is aware of multiple sustained or controlled release systems that are suitable for application to the present invention including OROS (Alza Corporation), described in U.S. Pat. No. 4,160,020 and other controlled release systems disclosed, for example, in U.S. Pat. Nos. 5,837,284, 6,183,778, 5,567,439, 6,042,847, and 7,083,808 the entire contents of which are herein incorporated by reference.

Administering CNS Stimulant(s) to Treat or Prevent Diseases Associated with ADHD

Genetic studies and family histories of patients with ADHD indicate that ADHD is often associated with chronic inflammation and may be the outcome of an inflammatory response, or a cause thereof. Reduced dopamine levels, which are associated with ADHD, are also associated with inflammation. Based on co-occurrences of ADHD and certain other diseases or conditions, many of which are also associated with inflammation, it is hypothesized that inflammation may be a root cause, and more likely an effect, in one or more of these conditions. Because chronic inflammation may be co-morbid in families and/or individuals with ADHD, and inflammation has been associated with other diseases and conditions, it is believed that treating ADHD with stimulants and optionally with anti-inflammatory and/or other agents to reduce inflammation and normalize catecholamines will also reduce the risk of developing one or more other conditions or diseases that are observed in ADHD individuals or families in which there are one or more ADHD individuals. Therefore another aspect of the invention relates to administering CNS stimulant(s) to individuals having ADHD and/or to immediate or extended family members of ADHD individuals as a means to treat and/or reduce the risk of developing such inflammation-associated diseases or conditions.

For example, increased levels of inflammation markers such as IL-1, IL-6 and TNF alpha are found in MS, Parkinson's disease, hypertension, diabetes, asthma, ulcerative colitis, Crohn's disease, celiac disease, psoriasis, dental diseases, endometriosis, migraine headaches, PMS, prostate cancer, breast cancer, and autoimmune diseases. Additionally, at region 2q12-13 on Chromosome 2 is a gene that is BCL-2 like 11. BCL-1 and BCL-2 are major causes of genetic breast cancer. There are also genes for Gastric cancer, resistance to malaria, Hepatocellular carcinoma, Cardiomyopathy, Thyroid disease, COPD, Autism, Colon cancer, Nonmedullary Thyroid Carcinoma, and susceptibility to coronary artery disease to name a few diseases. Beside this region, at 2q24, are genes for IDDM, NIDDM, and epilepsy. These diseases are all associated with inflammation. The gene for Von Recklinghausen's Disease or Neurofibromatosis Type 1 which can be found on part of chromosome 2p, but is also located on chromosome 17 at region 17q11.2. Granulocyte Colony Stimulating factor (G-CSF) is also found in this region at 17q11.2-q21. A gene for Autism (AUT6) is found at region 17q21. NOS2 is found at 17q11.2 and is involved in nitric oxide production, which impacts catecholamine levels, calcium channels, inflammation, sleep, blood flow and the formation of new memories. Additionally, a defect in this region could affect G-CSF, which may alter the expression of cytokines and nitric oxide synthetase. A defect in G-CSF or NOS-2 could alter inflammation and result in ADHD or Autism. Additionally, the serotonin transporter gene, SLC6A4, is found at region 17q11.2-q12 of Chromosome 17. SLC6A4 has been associated with OCD and the serotonin transporters are possible causes of ADHD. A defect in a serotonin transporter could increase serotonin and via the 5HT2, other serotonin receptors and second messengers lower dopamine, thereby causing inflammation. 17q24.2 also is associated with anticardiolipin antibodies along with other sites where ADHD genes are located. Two percent of the population has OCD and OCD is associated with ADHD. Psoriasis is another disease that originates from defects on chromosome 17. Region 17q21 also contains genes for vitamin B6 metabolism, Parkinson's disease, Picks disease, Supra Nuclear Palsy, dementia, renal cancer, Glioblastoma, Gastric cancer and ovarian cancer. G-CSF interacts with IL-10. IL-10 also interacts with IL-4 on chromosome 5 (a target for ADHD and schizophrenia). Lower levels of IL-10 are associated with Ulcerative colitis, Crohn's disease and IDDM (all diseases have elevated levels of IL-1, IL-6 and TNF alpha). IL-10 inhibits IL-1, IL-6, TNF alpha and other proinflammatory cytokines. Additionally, IL-10 has been shown to down regulate class II MHC complex expression. If G-CSF is mutated, this may then impact other cytokines, altering inflammation. G-CSF may be protective against Parkinson's disease and may treat Crohn's disease. If G-CSF function is lost, then inflammation will likely be altered. This may explain why 100% of people with Neurofibromatosis Type 1 get ADHD. However, it is likely that SLC6A4 is the defect that causes inflammation which may lead to “faulty” neurodevelopment and ADHD. Eventually, chronic inflammation and free radical production over a lifetime lead to other diseases that impact the entire body, not just the brain. NF1 may be linked with the gene that cause ADHD in some cases or share base pairs. It is interesting that both diseases likely cause inflammation in the gastrointestinal tract.

Another embodiment, relates to administering one or more CNS stimulant(s) and optionally co-administering one or more anti-inflammatory agent(s), at therapeutically effective serum levels substantially around the clock, preferably for about 24 and ½ to about 36 hours, more preferably for about 24 and ½ to about 26-27 hours or longer after the dose is administered. The concept is to treat or reduce the risk of developing, for example, cancers such as gastric cancer, hepatic cancer, colon cancer and thyroid cancer; cardiomyopathy, COPD, autism, spinocerebellar ataxia, dyslexia, hypercholesterolemia, inflammatory bowel disease, Crohn's disease, rheumatoid arthritis, Parkinson's disease and other diseases having an inflammatory component. In an illustrative embodiment, the method is applied to a patient having ADHD by administering one or more CNS stimulant(s) and one or more anti-inflammatory agent(s). Suitable stimulants and anti-inflammatory agents include those previously described herein pertaining to treating ADHD, including the stimulants methylphenidate and amphetamine products and dosage forms, and anti-inflammatory agents such as fish oil, DHA, EPA, pomegranate juice or extract, and others. Suitable dosages and dosage regimens for stimulant and anti-inflammatory agents are as previously described herein for treating ADHD. Alternatively and additionally, the anti-inflammatory agent may be administered once per day at a therapeutic dose.

Another embodiment of the invention relates to co-administering one or more CNS stimulant(s) with pyridoxal 5′ phosphate (P-5-P or PLP), together in a single dosage form, or separately as a treatment for a patient with ADHD and/or to reduce the risk of the patient developing a disease associated with ADHD and/or inflammation. Co-administration may be by any suitable means well known to the skilled artisan including simultaneous administration or sequential administration. It is believed that many ADHD patients are deficient in producing the active faun of vitamin B6 due in part at least to gastrointestinal inflammation and defects in synthesis of P-5-P that are genetically inherited with the gene(s) that causes ADHD. Defects in these enzymes appear to be part of the disease itself Inflammation can reduce absorption of amino acids and nutrients from the gastrointestinal tract which in turn adversely affects maintenance of normal catecholamine levels. Additionally, in some cases, treating ADHD may inhibit pyridoxal kinase and the production of P-5-P. By administering P-5-P with a stimulant, an adequate amount of P-5-P will be maintained to manufacture catecholamines, and run numerous other vital biochemical pathways in the body. Suitable dosages for P-5-P or PLP are in a range of about 25-50 mg to about 100 mg/day. In some cases 150 about mgs a day may be necessary.

Another embodiment of the invention relates to administering pyridoxal 5′ phosphate (P-5-P or PLP) alone or in combination with one or more anti-inflammatory agent(s) as a treatment for ADHD. When co-administered pyridoxal 5′ phosphate (P-5-P or PLP) and an anti-inflammatory agent(s) are administered together in a single dosage form, or separately. Co-administration may be by any suitable means well known to the skilled artisan including simultaneous administration or sequential administration. Suitable anti-inflammatory agents include synthetic as well as natural compounds including, but not limited to NSAIDs, fish oil, DHA, EPA, omega-3 fatty acids and pomegranate juice or extract. In one embodiment, the present method relates to co-administering a CNS stimulant with one or more natural product(s), for example, fish oil, omega-3 fatty acids DHA and EPA (available commercially as OMACOR®, Reliant Pharmaceuticals), or pomegranate juice or extract, grape seed extract, vitamin E, or the sulfated polysaccharide Fucoidan. Appropriate dosages of DHA and EPA are from about 650 mg to 2 grams per day; preferably from about 650 mgs to 1 gram per day. Appropriate dosages of pomegranate extract are from about 100 to 500 mg/day; preferably from about 200 to 250 mg/day. Appropriate dosages of grape seed extract are from about 100 to 500 mg/day; more preferably from about 200 to 400 mg/day, most preferably about 300 mg/day. Pyridoxal 5′ phosphate (available commercially as a vitamin supplement) is the active form of vitamin B6. It is believed that ADHD patients may be deficient in producing the active form of vitamin B6 due in part to biochemical defects in its synthesis and gastrointestinal inflammation. This type of inflammation reduces absorption and synthesis of amino acids from the gastrointestinal tract which in turn adversely affects maintenance of normal catecholamine levels. Lower levels may also be secondary to treatment with stimulants that exhibit feedback inhibition on pyridoxal kinase. Giving P-5-P would help a person manufacture their own neurotransmitters, but also run numerous critical biochemical pathways. Suitable dosages for P-5-P or PLP are in a range of about 25-50 mg to about 100 mg per day and may require up to about 150 mgs per day.

### Weekly Transdermal Administration

Certain illustrative embodiments also relate to once-weekly, or less frequent, transdermal administration of stimulant to treat ADHD. One embodiment, relates to once-weekly patch administration of a CNS stimulant, optionally also including an anti-inflammatory agent in which a suitable steady-state serum level of stimulant, for example methylphenidate or amphetamine, is maintained throughout the course of each day during a 3-7 day; preferably 4-7 day; more preferably 5-7 day; more preferably still 6-7 day; most preferably 7 day period. Alternatively, the method involves administering a stimulant via transdermal patch once per week to once every two weeks. Multiple topical application systems are known in the art that provide means for transdermal delivery of drugs including stimulant drugs.

The compounds may be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device for much of its flexibility. The material selected for the backing material should be selected so that it is substantially impermeable to the active agent and any other materials that are present, for example, the backing can be made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to co-administer a skin permeation enhancer with such drugs. Suitable enhancers are well known in the art. A suitable once per week patch delivery system is available from Alza Corporation as D-TRANS® Transdermal Technology.

The skilled artisan will be aware of an array of suitable techniques and methods for producing a once weekly patch delivery system for delivering pharmaceutically active agents including CNS stimulants, e.g. methylphenidate and/or amphetamine. Preferably the patch delivers from about 5 mg to about 30 mg of stimulant per 24 hour period. An appropriate dosage for treating ADHD by once weekly patch administration will depend on the response of the individual patient and the judgment of the treating physician. A suitable patch device would contain from about 20 mg to about 500 mg of stimulant in a reservoir, preferably from about 35 mg to about 300 mg of stimulant; more preferably from about 50 mg to about 200 mg; alternatively, greater than about 200 mg, or from about 200 to about 300 mg. The active stimulant agent would preferably also be mixed with or otherwise comprised of any number of controlled release substances, for example, ion exchange resins or amino acid polymers that would produce a delayed release of stimulant such that about 1 to 15% of the active agent is released per each 24 hour period. Suitable reagents and systems are disclosed in U.S. Pat. Nos. 4,931,279, 4,668,506 and 6,348,211, hereby incorporated by reference. It will be important that a patch maintain a steady state and when this is reached the rate of elimination of the medication equals the rate of absorption.

The present disclosure relates to methods and drug delivery systems for treating diseases involving inflammation, including Attention Deficit Hyperactivity Disorder (ADHD) by administering a CNS stimulant to a patient in need thereof so as to maintain steady state serum drug levels that remain therapeutically effective for about 24 and ½ to about 25-27 hours or longer after administration to allow the next dose to reach steady state to maintain an equal level of catecholamines for an individual. The method is employed to restore normal catecholamine levels throughout the day without over-stimulating or under-stimulating the patient. Unlike current recommendations, embodiments of the present invention and methods thereof are not limited to treating only if a patient is impaired. We would not wait for a person to lose their eyesight secondary to diabetes, the same needs to be considered with ADHD. The present method is surprising and unexpected given the conventional belief that people will not sleep on a stimulant. To the contrary, a vast majority of patients who take long acting stimulants according to the present invention sleep better. Blood pressure often drops, inflammation decreases, pain improves, PMS and migraines disappear, and sometimes people get off other medications. Their endocrine system is corrected and is not over-burdened, leading to premature failure. It is important to maintain the correct amount of medication substantially around the clock.

The present method is also different from current thinking that teaches dosing based on a patient's weight. Volume of distribution is only one factor in determining a therapeutically-effective dose. Absorption, metabolism, and genetic differences all play a role along with diet and activity level. Besides giving back only a level that a person needs to become normal, proper dosing can influence inflammation and the endocrine system, preventing other diseases that ADHD is associated with. Labs prior to and after treatment support this notion as do patient family medical histories and improvement in medical conditions. ADHD can now be correctly treated along with other psychiatric disorders. Hormones and interleukins are impacted. Only a small amount of neurotransmitters are found in the brain, the majority are found in the body. This explains why too much of an antipsychotic elevates prolactin, and why people speculate they cause prolactinomas, hypertension, diabetes and metabolic syndrome. This also explains why people can get Tardive Dystonia which looks like Parkinson's disease. Too much serotonin is equally as bad and can harm the cardiovascular system. The balance should be restored to normal and no more than that.

Additionally, providing P-5-P or PLP allows a person to make their own norepinephrine, dopamine and other neurotransmitters. Stimulants decrease the extra Dopamine transporters within one month of treatment as seen on a PET Scans. When a person can make their own catecholamines, ADHD control improves and people may be able to lower their dose of medication. Additionally, this allows other metabolic pathways to function normally such as amino acids that impact the entire body and brain. When elevated or depleted, amino acids are correlated with certain diseases. It is believed that the majority of ADHD patients have abnormal vitamin B6 levels prior to treatment with medication and that genes encoding enzymes involved in vitamin B6 synthesis or amino acid production are found in the same chromosomal regions as genes that are associated with ADHD as descried earlier. The presence of certain diseases in a patient, or member of the patient's family, is often predictive of which parent has ADHD. Amino acid profiles may also predict specific diseases that run in the family. For example, in one family, two siblings with ADHD had the same mother, but different fathers. Both children had the same vitamin B6 levels and the same amino acid profiles. Their mother and her mother both had ADHD. Thus, three generations on the same side of the family had ADHD and matching amino acid profiles.

The invention has been described with reference to various illustrative embodiments and techniques. However, it should be understood that many variations and modifications as are known in the art may be made while remaining within the scope of the claimed invention. The examples that follow are illustrative and are not intended to be limiting.

### EXAMPLE 1

### Treatment of 8 Year-Old Boy having ADHD

An 8-year old boy with ADHD is presently treated with 10 mg Adderall® twice per day, once in the morning before going to school, and a second dose while at school. The boy's parents report that he is disruptive and having other difficulties in the late afternoon and early evening. In addition to behavioral problems he is not able to fall asleep on his own, and for the past year has received Clonidine® 1 hour before bedtime. After a thorough medical exam, his treatment regimen is changed to 10 mg Adderall® XR given twice per day. The boy experiences a short-duration rise in blood pressure and heart rate, but vital signs return to normal within 2 days of changing dosage. After 1 week he is no longer having problems in the late afternoon and evening but he is feeling restless prior to bed and does not want to go to bed and stays up in his bed. He is then placed on Adderall XR three times a day as it is determined that the medication lasts 7 hours per dose. He now does well all day long and goes to bed without difficulty. Additionally, Clonidine is no longer needed. A sleep study shows a better quality of sleep with more stage III and IV sleep, less periodic limb movements, and better air flow. His parents report that he does not wet the bed anymore either. PET scans taken before and 6 months after the change in his treatment regimen reveal lower dopamine transporter levels in his striatum. The DATS return to normal levels within one month of treatment. He has also grown an inch.

### EXAMPLE 2

### Treatment of Adult Male with ADHD

A 30 year old male presents with feelings of anxiety, depression, and an inability to focus. He reports that he performed poorly in school though believed he was “smart.” After taking an in-depth medical and family history he is diagnosed with ADHD. He is slightly overweight, has a slight elevation in cholesterol, borderline hypertension, and some hypoglycemic episodes especially after a meal with a lot of sugar. He titrated up to 15 mg Focalin® XR three times a day, 300 mg grape seed extract and pomegranate extract. After 2 weeks of treatment he reports improvement in concentration ability and less anxiety and depression. His heart rate is decreased and his mean arterial blood pressure has decrease 20 points. He no longer experiences hypoglycemic episodes and his cholesterol has decreased. His appetite is normal, but he no longer has the desire to binge on sweets and has lost weight. The patient reports that this is the best he has felt in years and the best he has slept in 5 years.

### EXAMPLE 3

### Treatment of 28 Year Old Female

A 28 year old female presents with diagnosed ADHD and hypertension with a systolic pressure of 170 mmHg and a diastolic pressure of 100 mmHg. She has PMS and migraine headaches. She reports previous treatment with 20 mg regular release Ritalin® twice per day. She desires to return to school in order to graduate from college but is concerned about her prospects for success because she reports difficulties with focusing in the late afternoon. She fears that her inability to focus will negatively impact her performance at school. After a thorough medical history and exam she is titrated up to 20 mg Adderall® XR three times per day and twice daily intake of 1200 mg fish oil. After 8 weeks her blood pressure and heart rate are lower and she reports feeling more focused, efficient and able to sleep through the night. She reports less fluid retention and that her PMS and migraine headaches have gone away. After 6 months she continues to “feel better” and is a 4.0 GPA student at her local college. Her blood pressure is lowered to 110/70 and her pulse is also lower. She is able to cease taking Norvasc® and her blood sugar also improves along with occasional irritable bowel symptoms.

### EXAMPLE 4

### Family with History of ADHD, OCD and Cancer

The 5HT2A receptor has been associated with OCD, Seasonal Affective Disorder, Alcoholism, and a predisposition toward Schizophrenia. The gene for this receptor is found on chromosome 13. A female patient presents with OCD and ADHD. Two of her children also are diagnosed with OCD and ADHD. Her family history reveals that her father died from prostate cancer and two sisters were diagnosed with breast cancer. The patient has had a bilateral mastectomy secondary to breast cancer in her early 40s approximately four months after menopause. She tests positive for BRCA2 which is also found on chromosome 13 in the same region as the gene for the 5HT2A receptor.

Stimulation of 5HT2A with serotonin agonists has been shown to increase pro-inflammatory cytokines and lower catecholamines. Lowered catecholamines lead to slightly elevated prolactin and can alter GnRH. Elevated GnRH leads to spikes in estrogen and progesterone and may eventually lead to premature ovarian failure along with alterations in inflammation and co-factors. Elevated interleukins lead to increased free radical production and damage to DNA. Lowered dopamine would allow for angiogenesis. Elevated interleukins could induce CREB and increase the expression of ICAM and VCAM. BRCA2 lowers p53, p21 (tumor suppressor genes) as well as RAD51, which repairs damage to DNA. Catecholamines may interact with genes involved in cancer via common second messenger systems. Laboratory tests reveal that the patient has a high vitamin B6 level of 100, and an abnormal amino acid profile.

Treatment of the patient includes administering an extended release CNS stimulant at an appropriate dosage to achieve the desired therapeutic effect, substantially around the clock. Treating the patient's ADHD and correcting the catecholamine imbalance is expected to lower interleukins and free radicals and reduce other risk factors. It lowers GnRH and may interact with second messengers involved in cancer. She is placed on 100 mgs of P-5-P which corrects her amino acid profile and risk for developing other diseases associated with ADHD.

Additionally, the patient's children are screened for defects in 5HT2A and BRCA2. With early screening for ADHD and treatment with a CNS stimulant in accordance with the present invention it is expected that treatment will abrogate the harmful effects of pro-inflammatory cytokines, free radicals, and elevated hormone levels that might otherwise occur in the absence of treatment. This preemptive treatment of the patient's children is expected to reduce the risk of developing prostate, breast, or ovarian cancer later in life. Correcting her vitamin B6 deficit also eliminates risk factors. Helping her sleep also lowers cortisol and increases growth hormone levels, allowing for better functioning of her immune system.

Her husband also has ADHD and a family medical history significant for strokes, and heart attacks. He has a low level of proline and hydroxyproline along with a defect in vitamin B6. Hydroxyproline makes up connective tissue, collagen and lines arteries. When this is low people can develop plaques. One daughter has an amino acid profile identical to her father. Another daughter has a profile that is identical to her mother. The daughter that is identical to her mother also has an elevated vitamin B6 level and has bad PMS and GI issues like her mother. This daughter is treated around the clock with stimulant for her ADHD, given P-5-P, fish oil and antioxidants. She will get regular breast exams and ultrasounds. Her endocrine levels are normal and her amino acid profile returns to normal.

### EXAMPLE 5

### Treatment of 24 Year Old Male

A 24 year old obese male presents complaining of depression. He is on Lipitor for high cholesterol and has a heart rate of 116 and a blood pressure of 166/112. He also has “heart burn”. He is diagnosed with ADHD and titrated up to 15 mgs of Adderall XR three times a day. His heart rate decreases to 100 at the first visit four weeks later and after 8 weeks it is 88. Based on lab reports, his vitamin B6 level is elevated at 56 and he is deficient in arginine, ornithine, histidine, threonine, and taurine along with other amino acids. He also has an elevated IL-6 level. His vitamin D3 level is also very low. He is started on P-5-P and is supplemented with the aforementioned amino acids to help replenish his body. His taurine level is 18 and normal is at 54. He is a candidate for developing a cardiac arrhythmia.

He is supplemented for 4 months and then the supplements are stopped. His blood pressure and heart rate continue to decline and he begins to lose weight despite having a normal appetite. His cholesterol is lower and he does not have heart burn. His depression is gone and he is dreaming about becoming a lawyer. He remains on Adderall XR, however his dose is decreased to 10 mgs three times a day. He remains on P-5-P. The patient's amino acid profile is normal and his CRP is normal. CRP is an indirect measure of IL-6 and is impacted by low levels of P-5-P.

### EXAMPLE 6

### Stage 4 Ovarian Cancer

A 36 year old female presents with stage 4 ovarian cancer, skin picking, irritable bowel syndrome, PMS, allergies, and a tonsillectomy. She is BRCA1 positive and lost her mother in her early thirties due to breast cancer. Other family members have succumbed to breast cancer, pancreatic cancer, and colon cancer. She is screened for ADHD and is started on Adderall XR and titrated up to 15 mgs three times a day. She is tapered off Prozac for depression. Six weeks later she states that her depression is gone and her energy level is back to normal and her strength is retuning. Her nausea is also gone. Lab tests reveal her vitamin B6 is low so she is started on P-5-P. Her skin picking stops that same day. When she ran out of vitamin B6, her skin picking returns in one week, but stops the day she resumes taking vitamin B6.

Dopamine has been shown to inhibit VEGF and shrink ovarian cancer by 80% in 10 days in animal studies. Catecholamines may interact with genes involved in cancer in certain tissues. BRCA1 knocks p21, p53, and RAD51 out. Genes can be turned on and off based on signaling. Her amino acid profile fits with a defect in pyridoxine-5′-phosphate oxidase which is located on 17821.32. This is beside BRCA1 on chromosome 17. It is possible the patient has a defect in SLC6A4 not ADHD 2 on 17p11.2. 17p11.2 is the site of serine hydroxymethyltransferase. Additionally, it is possible that second messengers that are inactivated by BRCA-1 are responsible for lowering her catecholamines. Her amino acid profile also suggests that nitric oxide could be impacted as well, along with vitamin B6. Her Arginine was very low and her ornithine was extremely high, possibly secondary to a lack of P-5-P. She continues to progress and is returning to work. A CT scan reveals that a mass disappeared from an adrenal gland and after one dose of chemotherapy, her CA-125 drops by two thirds. This is the biggest drop her Oncologist has ever seen. It normally takes two to three doses to see a amall decrease in the level.

### EXAMPLE 7

### Once-Daily Long-Acting Stimulant Formulations

A sustained release dosage form is achieved using one of two means: capsule containing coated beads or a matrix tablet dosage form. Well-known industry excipients commonly accepted by the USP, EU and JP Pharmacopeias are used to produce extended release dosage forms. A release profile which will deliver the active moiety over substantially a 24 hour period is accomplished through the use of the matrix tablet. Both approaches utilize technology known to the skilled artisan.

Beads are manufactured using either an extrusion spheronization process containing the API or by applying the API via a coating solution to non pareils. Once the IR (immediate release) beads of API are defined, a sustained release coating is placed on top to control the dissolution of the API. Coatings consist of HPMC or ethylcellulose based polymers commonly used and accepted within the industry. An enteric coat maybe used to achieve the targeted profile. Beads have 3-4 different coat levels with distinctly different release profiles. In one embodiment, for example, the IR beads release API in 30 minutes while a coated bead may release at 5-6 hours, another at 9-10 hours and another at 14-16 hours. IR beads and beads with various levels of coating are mixed to achieve the desired release profile. IR beads are coated in a fluid bed system using a Wurster insert to achieve a uniform coating of the beads. Beads are filled into hard gelatin capsules to achieve a sustained release oral capsule dosage form.

IR beads (extrusion spheronization)

| Components | % w/w | Function |
|---|---|---|
| API | 15.00 | Active Pharmacologic Ingredient |
| Microcrystalline | 80.00 | diluent |
| Cellulose | | |
| HPMC E5 | 5.00 | binder |
| Purified Water | 30.00* | Granulating solution |

*Note: Removed during the drying process

IR beads (coating of non pareils)

| Components | % w/w | Function |
|---|---|---|
| API | 15.00 | Active Pharmacologic Ingredient |
| Non Pareil 16-20 | 80.00 | Diluent/ |
| PVP K29/32 | 5.00 | binder |
| Purified Water | 30.00* | Granulating solution |

*Note: Removed during the drying process

SR beads (coating IR beads)

| Components | % w/w | Function |
|---|---|---|
| API IR beads | 95/90/85.00 | Active Pharmacologic Ingredient |
| Ethylcellulose | 5/10/15 | SR coating |
| (Surelease ®) | | |
| Purified Water | 30.00* | Coating suspension |

*Note: Removed during the drying process

SR beads (coating IR beads)

| Components | % w/w | Function |
|---|---|---|
| API/beads | 85.00 | Active Pharmacologic Ingredient |
| HPMC (Eudragit | 8.5 | SR coating |
| RS 30D) | | |
| Triethyl citrate | 1.5 | plasticizer |
| Talc | 3.45 | Anti adherent |
| Purified Water | qs* | Coating suspension |

*Note: Removed during the drying process

Matrix tablet formulations use one of the following components as the matrix forming agent. Excipients such as carnuba wax, hydroxpropylmethyl cellulose, polyethylene oxide, and carboxypolymethylene are acceptable matrix forming agents. These excipients are used in conjunction with pore formers and lubricants required for manufacturing a sustained release tablet dosage form. A high shear granulation process or roller compaction process is implemented to achieve a suitable granulation. Granulated material will be compressed on a rotary tablet press to achieve the tablet hardness required to provide the targeted release profile.

Matrix Tablet

| Components | % w/w | Function |
|---|---|---|
| API | 15.00 | Active Pharmacologic Ingredient |
| HPMC K100 | 15.00 | Matrix former/binder |
| Microcrystalline | 60.00 | Insoluble diluent |
| cellulose | | |
| Lactose monohydrate | 9.00 | Soluble diluent |
| Magnesium stearate | 1.00 | lubricant |
| Purified Water | 30.00* | Granulating solution |

*Note: Removed during the drying process

### Theoretical Dissolution Profile

| | Time (hrs) | | | | | | | | | | |
|---|---|---|---|---|---|---|---|---|---|---|---|
| | 0 | 2 | 4 | 6 | 8 | 10 | 12 | 14 | 16 | 18 | 20 |
| Capsule | 0 | 15 | 25 | 30 | 40 | 50 | 60 | 70 | 80 | 90 | 100 |
| Tablet | 0 | 20 | 40 | 45 | 50 | 54 | 55 | 65 | 70 | 80 | 85 |

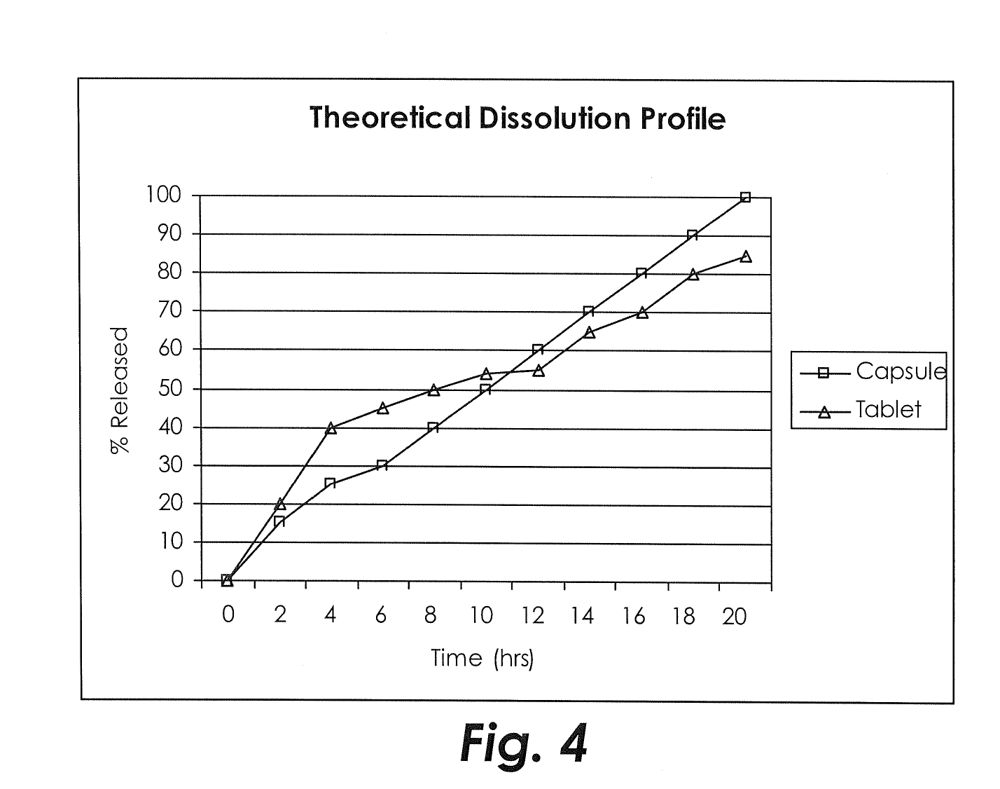

Formulations are evaluated in vitro using standard USP dissolution methods. Samples from the dissolution vessel are taken at the following time points, 1 hr, 3 hrs, 6 hrs, 9 hrs, 12 hrs, 15 hrs, 18 hrs, 21 hrs and 24 hrs. FIG. 4 shows a theoretical target dissolution profile for both capsule and tablet dosage forms. Once a dissolution profile is found which correlates with the targeted pharmacokinetics profile, it is then evaluated in vivo.

Suitable in vivo testing consists of animal models which are commonly used in the evaluation of sustained release dosage forms such as for example primates and canines. Using an animal model in conjunction with in-vitro dissolution will provide adequate information in the selection of the prototype formulation which will be tested in clinical bioavailability studies.

While the invention has been illustrated and described in detail in the foregoing drawings and description, the same is to be considered as illustrative and not restrictive in character, it being understood that only illustrative embodiments thereof have been show and described and that all changes and modifications that are within the scope of the following claims are desired to be protected.

1-33. (canceled)

34. A method for the treatment of ADHD in a patient in need thereof comprising administering a therapeutically effective amount of a CNS stimulant wherein said stimulant reaches a therapeutically-effective, steady state serum level that is maintained substantially around-the-clock.

35. A method as in claim 34 wherein said stimulant is administered orally one or more times daily.

36. A method as in claim 34 wherein said stimulant is administered orally once daily.

37. A method as in claim 34 wherein said stimulant is administered transdermally one or more times weekly.

38. A method as in claim 34 wherein said stimulant is administered transdermally once weekly.

39. A method as in claim 34 further comprising co-administering an agent selected from anti-inflammatory agent and pyridoxal 5′ phosphate.

40. The method of claim 39 wherein said agent is an anti-inflammatory agent selected from fish oil, DHA, EPA, GLA, pomegranate extract, NSAID, and grape seed extract.

41. The method of claim 39 wherein said agent is pyridoxal 5′ phosphate.

42. A method as in claim 34 wherein said steady state serum level comprises a substantially square-wave or pulsatile profile.

43. The method of claim 34 wherein said treatment results in clinical improvement of the patient's ADHD symptoms without increasing the risk of undesirable side effects.

44. A method for the treatment of ADHD as in claim 34 wherein said stimulant is administered once-daily in a sustained-release pharmaceutical dosage form comprising an immediate-release component and a delayed-release component such that said stimulant is substantially completely released from said dosage form over a period of about 23 to about 26 hours following administration.

45. A method of treating or reducing the risk of an ADHD associated inflammatory disease in a human subject at risk of or afflicted with such a disease comprising administering a therapeutically effective dosage of a CNS stimulant to said subject wherein said stimulant reaches a therapeutically-effective, steady state serum level that is maintained substantially around-the-clock wherein said treatment reduces inflammation without inducing adverse side-effects associated with stimulant use.

46. A method as in claim 45 wherein said disease is selected from the group consisting of cancer, cardiovascular disease, stroke, OCD, diabetes, arthritis, osteoporosis, asthma, COPD, Parkinson's disease, and multiple scelrosis, depression, migraine headache, Restless Leg Syndrome, obesity, peripheral vascular disease, macular degeneration, autoimmune disease, ulcerative colitis, and Crohn's disease.

47. A method as in claim 46 wherein said stimulant is administered once-daily.

48. A method as in claim 45 further comprising administering an agent selected from anti-inflammatory agent and pyridoxal 5′ phosphate.

49. The method of claim 48 wherein said anti-inflammatory agent is selected from fish oil, DHA, EPA, GLA, pomegranate extract, NSAID, and grape seed extract.

50. A sustained-release pharmaceutical dosage form for once-daily oral administration of a CNS stimulant comprising an immediate-release component and a delayed-release component wherein said stimulant is substantially completely released from said dosage form over a period of about 23 to about 26 hours following administration, and wherein upon administration to a patient in need thereof, said dosage form provides a steady state therapeutically effective serum level of stimulant substantially around-the-clock.

51. A sustained-release dosage form as in claim 50 wherein said immediate-release component releases stimulant within a period of about 30 minutes to about 4 hours following administration, and said delayed-release component begins releasing stimulant about 30 minutes to about 4 hours after administration.

52. A sustained-release pharmaceutical dosage form as in claim 50 further comprising an agent selected from an anti-inflammatory agent and pyridoxal 5′ phosphate.

53. A sustained-release transdermal patch for once-weekly administration of a CNS stimulant comprising an immediate-release component and a delayed-release component wherein said stimulant is substantially completely released from said dosage form over a period of about one week following application of said patch, and wherein said stimulant is maintained at a steady state therapeutically effective serum level in a patient substantially throughout said period.
